(19) United States
(12) Patent Application Publication
(10) Pub. No.: US 2023/0324376 A1
(43) Pub. Date: Oct. 12, 2023

(54) COMPOSITIONS AND METHODS FOR THE DETECTION AND MOLECULAR PROFILING OF MEMBRANE BOUND VESICLES WITH NANOPARTICLES

(71) Applicant: The University of Memphis Research Foundation, Memphis, TN (US)

(72) Inventors: Xiaohua HUANG, Memphis, TN (US); Thang Ba HOANG, Memphis, TN (US); Yongmei WANG, Memphis, TN (US); Caleb Edward GALLOPS, Memphis, TN (US)

(73) Assignee: The University of Memphis Research Foundation, Memphis, TN (US)

(21) Appl. No.: 18/132,746

(22) Filed: Apr. 10, 2023

Related U.S. Application Data
(60) Provisional application No. 63/329,779, filed on Apr. 11, 2022.

(51) Int. Cl. G01N 33/543; G01N 33/553; G01N 33/58; G01N 21/64; G01N 33/68

(52) U.S. Cl. CPC G01N 33/54346; G01N 33/553; G01N 33/587; G01N 21/6458; G01N 33/68; G01N 2021/8822

(57) ABSTRACT

The present disclosure featured compositions and methods related to the detection and molecular profiling of extracellular vesicles using optical probes, dual imaging approaches, and computationally programing-based image analysis methods. These compositions and methods leverage the unique optoelectrical properties of quantum dots, fluorescently labeled nanoparticles, and gold nanoparticles, which allow reliable, real-time detection of extracellular vesicles and vesicle surface bound or lumenal molecules at single vesicle level.

### CROSS-REFERENCE TO RELATED APPLICATIONS

This application claims the benefit of priority under 35 U.S.C. § 119(e) to U.S. Provisional Application No. 63/329,779, filed Apr. 11, 2022, which is incorporated herein by reference in its entirety.

### STATEMENT OF RIGHTS TO INVENTIONS MADE UNDER FEDERALLY SPONSORED RESEARCH

This invention was made with government support under Grant No. 1R15CA238890-01A1 awarded by the National Institutes of Health. The government has certain rights in the invention.

### BACKGROUND OF THE INVENTION

Cancer is a major public health issue worldwide. According to World Health Organization, cancer caused nearly 10 million deaths globally in 2020. In the United States, cancer is the second leading cause of death, with greater than 500,000 deaths annually. The major challenges in the fight against cancer are early detection and metastasis.

Growing research indicates tumor cells release exosomes to precondition organs for metastatic invasion, which suggests exosomes may have potential as biomarkers of cancer early detection and metastasis prediction. However, translation of exosomes into clinical biomarkers has been hampered due to the lack of facile and highly sensitive exosome molecular characterization methods capable of use in clinical settings.

### SUMMARY OF THE INVENTION

This disclosure provides compositions and methods related to the detection and profiling of extracellular vesicles (e.g., exosomes, microvesicles) at the level of single exosomes using nanoparticles and optical imaging. The present disclosure also features devices for detection and profiling of extracellular vesicles. In some aspects, this disclosure provides compositions and methods that leverage unique capabilities of nanotechnology and optical imaging to assess surface protein markers on individual exosomes from body fluid. In particular, provided herein are compositions and methods that combine high sensitivity gold nanoparticles (AuNPs) with optical imaging allowing for efficient exosome capture and rapid mask/target imaging. Accordingly, compositions and methods of this disclosure address technical barriers in molecular analysis of exosome surface markers to provide facile and highly sensitive exosome molecular characterization strategies for use in clinical applications and basic research.

In one aspect, this disclosure provides a method for characterizing membrane bound vesicles present in a biological sample, the method comprising: a) contacting a biological sample comprising a membrane bound vesicle comprising a lipophilic dye with a gold-coated substrate comprising a first capture molecule fixed to the surface of the substrate, wherein the first capture molecule specifically binds a first surface protein present on the surface of the membrane bound vesicle, thereby fixing the membrane bound vesicle to the surface of the substrate; b) contacting the membrane bound vesicle with a second capture molecule, wherein the second capture molecule is fixed to the surface of a nanoparticle, wherein the second capture molecule specifically binds a surface marker of interest on the membrane bound vesicle; c) subjecting the membrane bound vesicle to fluorescence imaging and dark field imaging, wherein the fluorescence imaging localizes the vesicles on the slide and the dark field imaging characterizes the presence or absence of the surface marker of interest on the vesicles, thereby obtaining dark field images and fluorescent images; d) computationally analyzing overlap of the fluorescence images and the dark field images to identify vesicles having or lacking the surface marker of interest; e) extracting pixel intensity of the nanoparticles from the images to characterize an expression profile of the protein of interest at a location of the vesicle, thereby obtaining a protein expression profile for the protein of interest present in the biological sample and quantifying target-specific vesicle subtypes.

In some embodiments, the method for characterizing membrane bound vesicles further comprises computationally analyzing the images and/or protein expression profiles to determine the fraction of vesicles that are positive or negative for the surface marker of interest and the level of expression of the surface marker of interest on the vesicles (e.g., positive vesicles or negative vesicles for the marker of interest).

In some embodiments of the method for characterizing membrane bound vesicles, the substrate is a glass microslide, silicon wafer, or other planar surface. In some embodiments, the first and/or second capture molecule is an antibody, aptamer, or other molecule that specifically binds an antigen present on the surface of an extracellular vesicle. In some embodiments, the second capture molecule is an antibody or antigen binding fragment thereof that specifically binds an ALIX, TSG101, CD81, CD63, or CD9 polypeptide.

In another aspect, this disclosure provides a method for characterizing exosomes present in a biological sample, the method comprising a) contacting a liquid biological sample comprising one or more exosomes comprising a lipophilic dye with a gold-coated multi-well slide comprising an antibody or antigen binding fragment thereof fixed to the surface of the slide, thereby fixing the exosomes to the surface of the slide; b) contacting the exosomes with a second antibody or second antigen binding fragment thereof fixed to a surface of a metal nanoparticle, wherein the second antibody or second antigen binding fragment thereof specifically binds a polypeptide surface marker of interest; c) subjecting the exosomes to fluorescence imaging and dark field imaging, wherein the fluorescence imaging localizes the exosomes on the slide and the dark field imaging characterizes the presence or absence of the polypeptide surface marker of interest on the exosomes, thereby obtaining dark field images and fluorescent images; d) computationally analyzing overlap of the fluorescence images and the dark field images to identify exosomes having or lacking the surface marker of interest; and e) extracting pixel intensity of the metal nanoparticles (e.g., gold nanoparticles) from the images to characterize an expression profile of a protein of interest at a location of the exosome, thereby obtaining the protein expression profile. In some embodiments, the protein expression profile and/or images are further computationally analyzed to determine the fraction of vesicles that are positive for the surface marker of interest and the level of expression of the marker of interest on marker positive vesicles and marker negative vesicles.

In various embodiments of the above aspects, or any other aspect of the invention described herein, the metal nanoparticle comprises silver, gold, copper, titanium, platinum, zinc, iron, or magnesium. In some embodiments, the metal nanoparticle is a gold nanoparticle. In some embodiments, the biological sample is blood, plasma, serum, cerebrospinal fluid, ascites, or culture media. In some embodiments, the polypeptide surface marker of interest is an ALIX, TSG101, CD81, CD63, or CD9 polypeptide. In some embodiments, the lipophilic dye comprises a lipophilic molecule having an alkyl chain and an affinity for a lipid bilayer of an extracellular vesicle. In some embodiments, a lipophilic molecule on the substrate comprises 1,2-distearoyl-sn-glycerol-3-phosphoethanoloamine conjugated polyethylene glycol thiol (DSPE-PEG-SH). In some embodiments, the lipophilic dye comprises cholesterol-polyethylene glycol-Cy5 (CLS-PEG-Cy5). In some embodiments, the fluorescence is generated using a laser. In some embodiments, the laser emits a wavelength of light between 600-700 nanometers. In some embodiments, the nanoparticle is bound to the vesicle or exosome via an antibody linked to the nanoparticle, wherein the antibody specifically binds to a marker present on the vesicle or exosome. In some embodiments, the vesicle or exosome comprises a polypeptide surface marker selected from the group consisting of HER2, CD44, CLDN4, EPCAM, CD151, LGALS3BP, HIST2H2BE, or HIST2H2BF. In some embodiments, detection of the marker is indicative of disease. For example, in some embodiments, the disease is selected from the group consisting of: cancer, neurodegenerative diseases, cardiovascular diseases, and pregnancy disorders. In some embodiments, the lipophilic dye comprises CLS-PEG-Cy5. In some embodiments, the second capture molecule or antibody is linked to the nanoparticle (e.g., metal nanoparticle) by NHS-PEG-SH. In some embodiments, the metal nanoparticle is between 10-100 nm in diameter. In some embodiments, the metal nanoparticle is about 60 nm in diameter.

In another aspect, this disclosure provides a dual fluorescence and dark field microscopic imaging system, the system comprising: a dark field microscope comprising a halogen illumination lamp configured for dark-field white light illumination and an objective lens positioned over a sample field; and an excitation laser positioned in an angled direction, wherein the angle is at least about 30-60 degrees relative to the sample field, wherein when a sample present in the sample field is illuminated by the lamp and excited by the laser, signals are transmitted to an imaging camera and a spectrometer. In some embodiments, the system comprises an excitation laser angled at about 45 degrees relative to the sample field. In some embodiments, the laser is a Griot continuous wave He laser.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “alteration” is meant a change (increase or decrease) in an analyte as detected by methods such as those described herein. In one embodiment, the alteration is in the level of a protein biomarker present on a membrane bound vesicle. As used herein, an alteration includes a 10% change in expression levels, preferably a 25% change, more preferably a 40% change, and most preferably a 50% or greater change in expression levels.

An “aptamer,” as used herein, refers to an oligonucleotide or polypeptide that specifically binds to a target molecule.

By “biomarker” or “marker” is meant an analyte the detection of which can be correlated with a particular physical condition or state. In some embodiments, the analyte is a polypeptide, polynucleotide, or a fragment thereof. The terms “marker” and “biomarker” are used interchangeably throughout the disclosure. For example, the presence and/or absence of biomarkers of the present invention can be used to detect a disease such as cancer, e.g., breast cancer. In some embodiments, an alteration in the level or structure of the marker is indicative of a disease state. Such biomarkers include, but are not limited to, molecules (e.g., proteins) present on the surface of extracellular vesicles.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “capture molecule” refers to a molecule that specifically binds a target. In particular embodiments, the capture molecule is polypeptide, polynucleotide, or small molecule. In one embodiment, the capture molecule is an antibody or aptamer that specifically binds a protein on the surface of an extracellular vesicle. In another embodiment, the capture molecule is biotin and the target is streptavidin.

By “detect” is meant to characterizing the presence, absence, or amount of an analyte in a sample.

By “detectable label” is meant a composition that when linked to a molecule of interest renders the latter detectable, via spectroscopic, photochemical, biochemical, immunochemical, or chemical means. For example, useful labels include metal nanoparticles, quantum dots, radioactive isotopes, magnetic nano- or micro-particles, metallic beads, colloidal particles, fluorescent dyes, electron-dense reagents, enzymes (for example, as commonly used in an enzyme-linked immunosorbent assay (ELISA), biotin, digoxigenin, or haptens.

By “disease” is meant any condition or disorder that damages or interferes with the normal function of a cell, tissue, or organ. Exemplary diseases that can be evaluated using a method of the invention include, but are not limited to, cancer, neurodegenerative diseases, cardiovascular diseases, and pregnancy disorders.

The term “exosome” refers to a cell-derived membrane bound vesicle comprising a membrane and lumen, wherein the exosome is typically between 30 and 200 nm in diameter. Exosomes, like other membrane bound vesicles, may contain surface bound or lumenal molecules that allow the identification of the cell type from which the exosome originated.

By “extracellular vesicle” is meant a membrane bound vesicle that is present extracellularly. Exemplary extracellular vesicles include exosomes and microvesicles.

By “fluid-tight” is meant capable of retaining a liquid sample for the duration of an assay without the sample leaking or seeping from a compartment or well.

By “fluorescent probe” or “fluorescent label” is meant any reporter molecule that emits a detectable fluorescent signal when in an excited state.

By “fluorescently labeled nanoparticle” is meant a nanoparticle (e.g., a bead or metallic particle) that is labeled (e.g., conjugated, coated, or otherwise attached) with a fluorescent moiety (e.g., a fluorescent dye).

The term “lumenal molecule” refers to a molecule residing within the lumen of a membrane bound vesicle.

By “membrane bound vesicle” is meant any small fluid filled sac having a lipid bilayer that surrounds it. Exemplary membrane bound vesicles include, but are not limited to, extracellular vesicles (e.g., microvesicles and exosomes) and apoptotic bodies. A membrane bound vesicle may comprise an exosome, and when the membrane bound vesicle fuses with the plasma membrane of a cell, it releases the exosome into the extracellular milieu.

By “membrane tag” is meant a composition comprising a lipophilic moiety bound to a linker bound to a moiety that can bind to or otherwise sequester a quantum dot. For example, a membrane tag may have a biotin moiety that is able to bind the streptavidin moiety of a streptavidin-labeled quantum dot. In one embodiment, the lipophilic moiety is cholesterol, the linker is PEG, and the moiety that binds the quantum dot is biotin.

By “metal nanoparticle” is meant a particle of between 1 to 200 nanometers in size with light scattering properties that render the particle detectable by dark-field microscopy methods. In some embodiments, the metal nanoparticle is a gold nanoparticle (AuNP).

The term “microvesicle” refers to vesicles originating from the plasma membrane of a cell. Microvesicles are typically larger than exosomes.

By “primary antibody” is meant an antibody having an affinity for an epitope on a molecule of interest.

“Quantum dot” (QD) refers to a probe made from nanocrystals of semiconductor material, wherein the probe can emit electromagnetic radiation. Generally, larger diameter quantum dots emit longer wavelength radiation than smaller diameter quantum dots. For example, quantum dots having a diameter of about 2 nm will have a visible emission farther in the blue region than a quantum dot having a diameter of about 5 nm. A quantum dot can also be tuned to emit a particular wavelength by changing the composition comprising the quantum dot or the structure thereof without changing the crystal size.

By “reduce” is meant a negative alteration of at least 10%, 25%, 50%, 75%, or 100%.

By “reference” is meant a standard or control condition. In some embodiments, a reference is the level of an analyte, marker, or other readout present in a control cell (e.g., an untreated cell, wild-type cell).

As used herein, “secondary antibody” refers to an antibody having an affinity for an epitope on a primary antibody. In some embodiments, a secondary antibody can comprise a label (i.e., a quantum dot).

By “subject” is meant a mammal, including, but not limited to, a human or a non-human mammal, such as a bovine, equine, canine, ovine, porcine, feline, or other domesticated mammal.

A “surface bound molecule” refers to a molecule that is fixed to or integrated into the membrane of a membrane or other surface.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1C are schematic illustrations of the biogenesis, molecular composition, and heterogeneity of exosomes. FIG. 1A is a schematic of the formation of exosomes via exocytosis. FIG. 1B is a schematic of the composition of exosomes, including lipids, nucleic acid (e.g., mRNA, miRNA, tRNA, rRNA, and DNA), and proteins (e.g., tetraspanins, receptors, adhesion molecules, transporters, cytosolic and cytoskeleton proteins). FIG. 1C illustrates exosomal heterogeneity in biofluid of a cancer patient. The exosomes consist of exosomes from tumor (shown in dark gray); exosomes from non-tumor tissue (shown in light gray); and exosomes from hematopoietic cells (shown in gray).

FIGS. 2A-2H illustrate an overview of the dual imaging single vesicle technology (DISVT) for single exosome surface protein profiling. FIG. 2A is a schematic showing the principle of DISVT. FIG. 2B is a schematic showing dual fluorescence imaging and dark field imaging using one single customized optical microscope. FIG. 2C is a comparison of the absorption spectrum of 60 nm gold nanoparticles (AuNPs), the absorption spectrum of fluorescence membrane dye, cholesterol-polyethylene glycol-Cy5 (CLS-PEG-Cy5, PEG molecular weight=2000 Da) and the fluorescence spectrum of CLS-PEG-Cy5. The AuNPs exhibit the localized surface plasmon resonance (LSPR) around 520 nm. The CLS-PEG-Cy5 exhibits the absorption peak around 640 nm and fluorescence peak around 655 nm. FIG. 2D shows a transmission electron microscope image of the 60 nm AuNPs. FIGS. 2E-2H show an example of dual imaging and protein profiling using DISVT. FIG. 2E shows a fluorescence image of exosomes captured from the plasma of a stage III human epidermal growth factor receptor 2 (HER2)-positive breast cancer patient. FIG. 2F is the dark field image of FIG. 2E after labeling with HER2 targeted AuNPs. FIG. 2G is the superimposed image of FIG. 2E and FIG. 2F that shows the AuNP-bound exosomes (white boxes) and AuNP-free exosomes (light grey boxes). FIG. 2H is the population density histogram of the plasma exosomes from the stage III HER2-positive breast cancer patient that shows the distribution of AuNP-bound exosomes (dark grey peak) and AuNP-free exosomes (light grey peak). Analysis of the exosome population density histogram derives the fraction of AuNP-bound and AuNP-free exosomes over the total captured exosomes. The fraction of HER2-positive exosomes over the captured exosomes is calculated from the AuNP-bound exosomes after correction with nonspecific binding using AuNPs linked with isotype IgG.

FIGS. 3A-3B show a multi-well Au chamber slide for exosome capture and detection. FIG. 3A is a photographic picture of an Au-coated glass slide. FIG. 3B is a photographic picture of the Au chamber slide formed by assembly of a multi-well cassette with the Au-coated glass slide.

FIG. 4 illustrates the generation of thiol-functionalized antibodies, which are obtained by incubating antibodies with N-hydroxysuccinimide-poly(ethylene glycol)-thiol (NHS-PEG-SH, PEG molecular weight=1000 Da) for 2 hours at 37° C., followed by purification using a 10K centrifugal filter.

FIG. 5 illustrates the preparation of target-specific antibody-conjugated AuNPs. The preparation involves two steps: (1) binding of thiol-functionalized detection antibodies to AuNPs and (2) saturating and stabilizing the AuNPs with methoxy-PEG-SH (mPEG-SH, molecular weight=5000 Da).

FIG. 6 is a schematic of exosome capture and labeling. In a typical procedure, exosomes are captured directly from a diluted biofluid sample such as plasma onto a multi-well Au chamber slide that is functionalized with a capture antibody targeting an exosome marker such as CD81. Surface protein markers of interests are labeled with target-specific antibody-conjugated AuNPs (typically 50 pM for 1 hour at room temperature), followed by exosome membrane labelling with fluorescence membrane dye such as CLS-PEG-Cy5 (typically 20 μM for 15 min at 37° C.).

FIG. 7 is a photographic picture of the dual dark field and fluorescence microscopic imaging system. The system is built on a customized Nikon LV 150N microscope. The microscope allows for bright/dark field imaging with a halogen illumination lamp. Fluorescence imaging is accomplished with a red diode laser in an angled direction (˜45 degree relative to the sample surface) from the side of the objective lens.

FIGS. 8A-8E illustrate the labeling, superimposing, and signal extraction with the single extracellular-vesicle dual imaging analysis (SEDIA) method using plasma exosomes from a stage III HER2-positive breast cancer patient as an example. FIG. 8A shows a fluorescence image of the plasma exosomes labeled with HER2/AuNPs. FIG. 8B shows a dark field image of plasma exosomes labeled with HER2/AuNPs. FIG. 8C shows the superimposed image of FIG. 8A and FIG. 8B. In the superimposed image, AuNP-bound exosomes were marked as white boxes and AuNP-free EVs were marked as light grey boxes. FIG. 8D shows the exosome population density histograms extracted from the superimposed images using the HER2/AuNPs showing AuNP-bound exosomes (dark grey peak) and AuNP-free exosomes (light grey peak). FIG. 8E shows a fluorescence image of the plasma exosomes labeled with IgG/AuNPs. FIG. 8F shows a dark field image of plasma exosomes labeled with IgG/AuNPs. FIG. 8G shows the superimposed image of FIG. 8E and FIG. 8F. In the superimposed image, AuNP-bound exosomes were marked as white boxes and AuNP-free EVs were marked as light grey boxes. FIG. 8H shows the exosome population density histograms extracted from the superimposed images using the IgG/AuNPs showing AuNP-bound exosomes (dark grey peak) and AuNP-free exosomes (light grey peak).

FIGS. 9A-9E illustrate an example of DISVT for early cancer detection. FIGS. 9A-9D show the population density histograms of plasma exosomes from different subjects labeled with HER2/AuNPs (dark grey) and IgG/AuNPs (light grey). FIG. 9A shows the population density histograms of plasma exosomes from a stage I HER2-positive breast cancer patient. FIG. 9B shows the population density histograms of plasma exosomes from a stage III HER2-positive breast cancer patient. FIG. 9C shows the population density histograms of plasma exosomes from a healthy donor. FIG. 9D shows the population density histograms of plasma exosomes from a stage III HER2-negative breast cancer patient. FIG. 9E shows the box plot of FHER2 for healthy donors (n=10), early-stage HER2-positive breast cancer patients (n=10), stage III HER2-positive breast cancer patients (n=10), and stage III HER2-negative breast cancer patients (n=10). The results showed that the DISVT detected the HER2-positive breast cancer at both early-stage and stage III.

FIGS. 10A-10B show the results for exosomal protein detection with enzyme-linked immunosorbent assay (ELISA). FIG. 10A shows the HER2 expression level of plasma exosomes of different subjects by ELISA. FIG. 10B shows the box plot of HER2 expression level for patient and healthy controls of FIG. 10A. The results show that ELISA can only detect the stage III HER2-positive breast cancer.

FIGS. 11A-11C show a comparison of the performance of DISVT and ELISA for breast cancer detection. FIG. 11A shows the receiver operation characteristic (ROC) curves of DISVT and ELISA between healthy control and early-stage HER2-positive breast cancer patients. FIG. 11B shows the ROC curves of DISVT and ELISA between healthy control and stage III HER2-positive breast cancer patients. FIG. 11C shows the ROC curves of DISVT and ELISA between early-stage and stage III HER2-positive breast cancer patients. The results show that DISVT exhibit much higher area of under curve (AUC) values and thus much higher sensitivity than ELISA for the detection of HER2-positive breast cancer at both early-stage and stage III as well as to differentiate early-stage and stage III HER2-positive breast cancer.

### DETAILED DESCRIPTION OF THE INVENTION

This disclosure features compositions and methods to detect and characterize extracellular vesicles (e.g., exosomes and microvesicles) by labeling and detecting biomarkers present on the vesicles with nanoparticles and detecting the nanoparticles in a dual imaging approach.

The disclosures herein are based, at least in part, on the discovery of imaging strategies in which nanoparticles are used as a contrast agent to enhance detection of biomarkers, including biomarkers present at low levels, on single extracellular vesicles which are localized by elastic light scattering imaging or fluorescence imaging with fluorescence labels.

As reported in detail below, the disclosure provides a facile and rapid single vesicle imaging method to quantitatively characterize disease-associated exosome populations for medical diagnostics and monitoring as well as basic biomedical research. The method captures exosomes directly from diluted plasma samples onto a palm size multi-well sample slide and then uses a facile yet novel dual optical imaging method to localize individual exosomes and detect targeted surface protein markers on the individual exosomes. Using an automatic imaging analysis algorithm, expression profile of targeted protein markers is determined within 2-3 minutes. An analysis of the protein expression profile determines the fraction of exosome subpopulations that are positive for the targeted protein markers. The quantitative protein expression profile and the fraction of protein-positive exosome populations have implications to detect cancer at early-stage, predict metastasis, monitor disease progression and cancer recurrence. They can also be used for basic vesicle research to understand cancer biology. The methods overcome barriers in current exosome molecular analysis in terms of sensitivity, efficiency, simplicity, and sample consumption. The novel dual imaging method allows detection of low number of antigens on single vesicles. The method can process over 100 samples on a single palm size chamber slide, with potential expansions to several hundred samples with further automatic development on sample deposition. It analyzes plasma samples directly, without complicated and time-consuming plasma processing that are often used in current technologies. Signal collection for each sample only takes seconds. Each test only requires few microliters of more than 10 times diluted plasma. This transforming molecular exosome technology can be translated into an inexpensive new generation liquid biology for routine cancer screening and monitoring with easily accessible blood samples.

Because biomarkers can reflect the origin of the extracellular vesicles, the methods and compositions described herein can be used to identify the presence or absence of extracellular vesicles that originate from diseased cells. As such, methods and compositions described herein can be used to detect disease without time consuming processes such as the pre-isolation of the extracellular vesicles.

In one aspect, this disclosure relates to a high sensitivity dual mask and target imaging approach for characterizing extracellular vesicles. A membrane dye is used to localize extracellular vesicles with fluorescence (mask) imaging. AuNPs are used to detect biomarkers with dark field (target) imaging. Due to their strong light scattering properties, AuNPs are detectable at single particle level with simple dark field microscope. This offers the possibility to detect targeted surface protein markers on individual exosomes at single molecule level. Furthermore, because surface protein markers are detected using dark filed imaging, compositions and methods described herein can assess extracellular vesicles while reducing the negative effects of photobleaching.

Compositions and methods described herein can make use of a simple compact microscope for simultaneous mask and target imaging by coupling fluorescence with the dark field microscopy to assess exosome populations. In some embodiments, the light emitting device (e.g., a laser or mercury arc lamp) is configured so as to emit light onto a target sample at an angle with respect to an objective lens, which allows for large area (micrometer scale) of sample imaging at single exosome resolution, while also reducing effects of photobleaching. In some embodiments, methods of the disclosure can be carried out with a microscopic device costing as low as $75,000, which is nearly three times cheaper than a confocal microscope (>$200,000). Another aspect of the disclosure is the fast single extracellular-vesicle dual imaging analysis (SEDIA) method, which is a python-based algorithm that can rapidly analyze the mask and target images within minutes, as compared to 1-2 hours with conventional ImageJ software. The image analysis determines the expression profile of protein markers on individual exosomes and calculate the fraction of exosome subtypes to detect and monitor disease signals.

Currently reported technologies in the literature for single exosome surface protein profiling include flow cytometry, fluorescence imaging, and proximity barcoding assay. In flow cytometry, exosomes are purified from plasma by ultracentrifugation or other isolation methods, labeled with fluorophore-labeled antibodies, and detected by nanoflow cytometry or imaging flow cytometry. However, analysis of exosomes with flow cytometry remains hassle due to the small size (30-200 nm) of exosomes and low signal-to-background noise. In the fluorescence imaging method, one method is to capture purified exosomes onto functionalized microfluidic chips, labeled with rounds of fluorophores and detected with a fluorescence microscope. This microfluidics-based method is complicated, impractical for clinical application for large amount of samples or real-time monitoring. It is not able to detect exosomes with low level of marker expression due to the limited sensitivity of organic fluorophores. Another way to detect single exosomal proteins based on fluorescence imaging is to use advanced microscopes such as stochastic optical reconstruction microscopy (STORM), which is high cost ($200,000 and above). A third way to detect proteins on single exosomes with fluorescence imaging is amplify fluorescence signals by rolling circle amplification (RCA) reaction. For this amplification-based method, the sample preparation is complicated, and it requires expertise and skill in designing nucleic acid-based probes. The barcoding assay profiles surface proteins on single exosomes using DNA-protein conjugates and next-generation sequencing, which is very sophisticated and expensive. It is thus not practical for routine use as a screening and monitoring technique for general public.

Compared to these existing methods that have been reported in the literatures, the dual imaging technologies described herein have the following major advantages: 1) simple sample preparation, following a single three-step process (exosome capture, labeling, and imaging) with no need to purify exosomes; 2) low sample consumption as each sample requires only 10 microliters or less of >10 times diluted plasma; 3) fast data collection and analysis, collecting images within seconds and processing multiple images simultaneously within seconds; 4) high sensitivity, as surface protein markers as low as a single molecule on single exosomes can be detected by the AuNP probe under dark field imaging; and 5) cost-effective instrumentation, being able to be performed with a regular optical microscope with dark field and fluoresce imaging modalities ($50,000 or less). In some embodiments, samples will be analyzed on a multi-well chamber slide. In some embodiments, sample preparation is automated such that the throughput on the chamber slide can reach over 200 wells, which allows simultaneous analysis of over 100 samples.

Characterization of exosome populations according to this disclosure can be useful for predicting or diagnosing disease, monitoring a treatment, assessing treatment efficacy, and basic biomedical research. In some embodiments, this disclosure provides compositions and methods capable of assessing exosomes directly from patient samples. In some embodiments, the exosomes are captured directly from patient fluid samples (e.g., a diluted plasma sample) onto a palm size multi-well sample slide. The multi-well sample slide is then subjected to dual optical imaging analysis to localize individual exosomes and detect targeted surface protein markers on the individual exosomes.

In some embodiments, compositions and methods of the present disclosure make use of an automated imaging analysis algorithm to rapidly generate an expression profile of targeted protein marker. Such a profile can be generated within, for example, 1-3 mins. Analysis of the protein expression profile reveals the fraction of exosome subpopulations that are positive or negative for the targeted protein markers. The quantitative protein expression profile and the fraction of protein-positive, or protein negative, exosome populations provide clinically relevant information with implications for disease, e.g., cancer, including early-stage detection, metastasis, progression, and recurrence. Compositions and methods disclosed herein can also be used for basic vesicle research to understand cancer biology.

The compositions and methods described herein overcome barriers in current exosome molecular analysis by providing high sensitivity, efficiency, simplicity, and low sample consumption. The novel dual imaging method provided by certain embodiments of the present disclosure allows detection of low number of antigens on single vesicles. The method can process over 100 samples on a single palm size chamber slide. For example, compositions and methods described herein can be used to process anywhere between 1 to 1000 samples, or more, on a single slide. Furthermore, compositions and methods described herein can be expanded to process tens to hundreds of fold more samples with automated sample deposition. In some embodiments, methods and compositions described herein can be used to analyze patient samples, e.g., plasma samples, directly without complicated or time-consuming processing, e.g., plasma processing, that are often used in current technologies. Signals from patient samples are collected rapidly. For example, signal collection from a patient sample can occur within seconds, e.g., within 1-3 seconds, providing clinicians with rapid information to guide important clinical decisions. Moreover, compositions and methods described herein are highly sensitive, as such, each test requires minimal sample (e.g., a few microliters of more than 10 times diluted plasma). Thus, biological samples that are difficult or painful to obtain are minimized. Accordingly, compositions and methods described herein can provide rapid, inexpensive approaches for routine disease screening and monitoring.

### Extracellular Vesicles

Extracellular vesicles are lipid-delimited particles that are naturally released from almost all types of cells and, unlike a cell, cannot replicate. In some embodiments, the extracellular vesicles are exosomes. Exosomes circulate in blood and many other body fluids, with typical concentration of 109-1011 vesicles per milliliter of blood. The level and molecular profile of circulating tumor exosomes have been shown to correlate with tumor burden, and tumor-derived exosomes can transfer oncogenic factors to other cells to promote tumor growth and progression. Exosomes are generally stable and can tolerate multiple cycles of freezing and thawing while preserving structure and molecular contents for over five years when stored in liquid nitrogen. Thus, exosomes offer a robust source to discover blood-based biomarkers for clinical use.

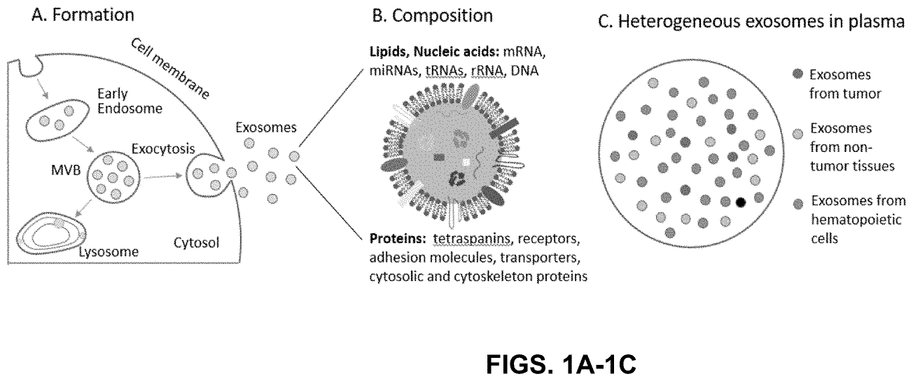

Exosomes originate in a cell and are eventually released directly from the cell into the extracellular milieu, or they are released when the membrane bound vesicle in which they reside fuses with the plasma membrane of the cell (FIG. 1A). Referring to FIG. 1B, an exosome may comprise proteins, carbohydrates, lipids or other molecules on its surface (such as embedded in its lipid bilayer membrane). An exosome may also contain lipids, carbohydrates, proteins or nucleic acids in its lumen. In some instances, the nucleic acids in the lumen of the exosome include mRNA, miRNA, tRNA, rRNA, and DNA nucleic acids and proteins reflective of the cells of origin. In some embodiments, the proteins in the lumen of the exosome or associated with the exosome's membrane include, but are not limited to, tetraspanins, receptors, adhesion molecules, transporters, cytosolic proteins, and cytoskeleton proteins.

Furthermore, extracellular vesicles, e.g., exosomes, have been implicated as important mediators of intercellular communication whereby they promote many pathophysiological disorders including cancer, neurodegeneration, and inflammatory disease. Because tumor cells continuously release exosomes, molecular analysis of exosomes in body fluids can detect cancer at early-stage. Growing research shows that tumor cells send out exosomes to precondition distant organs for metastatic invasion. Particularly, studies with animal models revealed that tumor-derived exosomes with distinct integrins prepared organ-specific pre-metastatic niches in breast cancer. The exosomal integrins fused with target cells activating them in a tissue-specific fashion to direct organ-specific colonization and thus initiated pre-metastatic niche formation. These findings pointed to the possibility that the integrin profiles of plasma exosomes from cancer patients could be used to detect impending breast cancer metastasis and organotrophic dissemination. Despite the great clinical potential, translation of exosomes into clinical practices is hampered by lack of facile and highly sensitivity technologies for detection and real-time analysis of tumor derived exosomes in plasma. The technical barriers are the small size of exosomes (less than 200 nm) and contamination of tumor-derived exosomes by vast amount of non-tumor exosomes from various tissues and hematopoietic cells (FIG. 1C).

The molecules on the surface or in the lumen of an exosome can be indicative of the type of cell from which the exosome originated. Molecules associated with a disease may be excreted from a diseased cell in or on an exosome or other extracellular vesicles, and detection of such exosomes or other extracellular vesicles comprising the disease-associated molecules is evidence of a disease-state. In some instances, the molecule associated with a disease may be a marker for the disease. In other embodiments, the molecule is associated with a disease only when it is present in amounts that differ from a reference amount associated with a healthy state. For example, in some cases overexpression of a molecule that is present in healthy cells is indicative of disease. In some embodiments, a sample taken from a subject may comprise a heterogenous population of exosomes. Referring to FIG. 1C, plasma from cancer patients may contain exosomes derived from tumor cells, non-tumor cells, and hematopoietic cells.

In some embodiments of the present disclosure, extracellular vesicles can be isolated or captured by a lipophilic capture agent. In other embodiments of the present disclosure, extracellular vesicles can be isolated or captured using an antibody having an affinity for the particular molecule on the surface of the extracellular vesicle to bind to the molecule. Exosomes contain several proteins that differentiate them from other types of extracellular vesicles including microvesicles and apoptotic bodies. Furthermore, surface proteins on exosomes carry information about their tissues of origin. Proteins that differentiate exosomes from other types of vesicles and apoptotic bodies include ALIX, TSG101, and tetraspanins CD81, CD63, and CD9. The tetraspanins have been widely used as exosome markers to capture exosomes as they are found in a significant amount of exosomes from many different origins. For example, CD81 can be used as the exosome marker to capture exosomes. Other exosome markers such as CD9 and CD63 can also be used. The capture antibody having an affinity for the proteins associated with an exosome (or other extracellular vesicle) may be tethered to a substrate or bound to a second molecule (e.g., magnetic beads) that allows for isolation of the capture antibody-extracellular vesicle complex. For example, in some embodiments of the present disclosure, the capture antibody is tethered to a surface of an array. In some embodiments the capture antibody is a monoclonal antibody. In some embodiments, the antibody is a polyclonal antibody. When the antibody specific to tetraspanins is used, exosomes do not need pre-purification. Exosomes are diluted with phosphate buffer solution (PBS), filtered by size exclusion, and used directly for capture and detection.

In some embodiments of the present disclosure, the exosomes are captured in a chamber slide. In some embodiments, the chamber slide contains a glass slide coated with Au film and a 3D printed array cassette. In some embodiments, the device contains over 100 wells for simultaneous multiple analyses.

In some embodiments of the present disclosure, an integrated single particle microscope is used to image exosomes. In some embodiments, a confocal fluorescence microscope is used. In some embodiments, a fluorescence microscope is used to examine the existence of exosomes. In some embodiments, the exosome or other extracellular vesicle is labeled with a lipophilic dye (e.g., CLS-PEG-Cy5) so that the exosome can be detected by fluorescence imaging.

### Metal Nanoparticles (e.g., AuNPs)

Certain aspects of the disclosure make use of bioimaging techniques to visualize markers associated with disease. In some embodiments, a metal nanoparticle is used to label biomarkers (e.g., surface proteins) of interest (e.g., cancer markers) on the extracellular vesicle membrane or within the lumen of the extracellular vesicle. The metal nanoparticle can comprise silver, gold, copper, titanium, platinum, zinc, iron, or magnesium nanoparticle. In some embodiments, due to a strong localized surface plasmon resonance and light scattering properties, the metal nanoparticle is a gold nanoparticle.

In some embodiments, the metal nanoparticles used in compositions and methods of the disclosure are uniform in size. In some embodiments, the nanoparticles have a diameter of 20, 30, 40, 50, 60, 70 or 80 nanometers. In some embodiments, the nanoparticles have a diameter of 60 nanometers. In some embodiments, the metal nanoparticles are substantially spherical. In some embodiments, the metal nanoparticles comprise a non-spherical shape. In some embodiments, compositions and methods of the disclosure make use of one or more different shapes of nanoparticles to differentially label distinct biomarkers. For example, in some embodiments, a first surface protein is labeled with a spherical metal nanoparticle, and a second surface protein is labeled with a non-spherical metal nanoparticle.

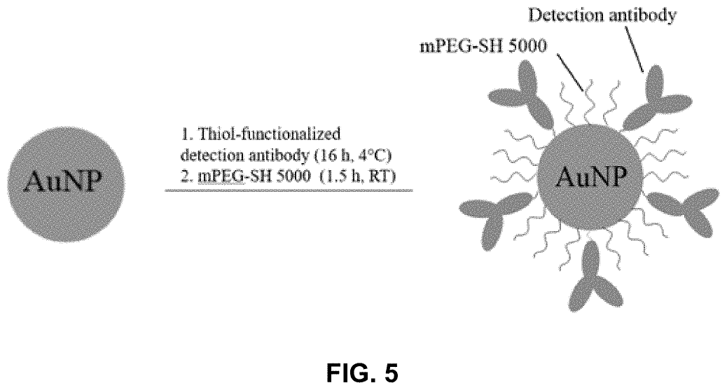

In some embodiments, a metal nanoparticle is coupled to an antibody, or a fragment thereof, which facilitates the binding of the metal nanoparticle to the biomarker of the extracellular vesicle. In some embodiments, coupling the antibody or the fragment thereof to the metal nanoparticle comprises two steps. For example, making reference to FIG. 5, preparing the metal nanoparticle can include binding antibody-PEG-SH to the metal nanoparticles and then saturating the metal nanoparticle with mPEG-SH 2000. The antibody can specifically bind to a biomarker of interest. In some embodiments, the biomarker is a marker associated with disease, for example, cancer. For example, in some embodiments, the antibody linked to the metal nanoparticle has a binding affinity for HER2, EpCAM, CD44, CLDN4, EPCAM, CD151, LGALS3BP, HIST2H2BE, or HIST2H2BF, which are associated with cancer.

In some embodiments, one or more QD probes are used to label an exosome's membrane. The one or more QD probes can be used in combination with the metal particles described above. The one or more QD probes may comprise different wavelengths. Using two different QD probes (two-color imaging) in conjunction with a laser microscopy system or a confocal microscope and high throughput chamber slide, or array, a panel of cancer-relevant protein markers on and inside exosomes can be screened by both spectroscopic bulk measurements and by imaging-based single exosome analysis. In some embodiments, the exosome membrane is not labeled, and exosomes are imaged directly in dark field to localize exosomes.

In some embodiments of the present disclosure, a magnetic nanoparticle coated with fluorescent dye is used to label proteins of interest (e.g., cancer markers) on the exosome membrane or within the lumen of the exosome. In some embodiments, a second dye is used to label an exosome's membrane. The visible signals emitted from the first and second dyes are different wavelengths. Using two different dyes (two-color imaging) in conjunction with metal nanoparticles and a laser microscopy system or a confocal microscope and high-throughput chamber slide, or array, a panel of cancer-relevant protein markers on and inside exosomes can be screened by both spectroscopic bulk measurements and by imaging-based single exosome analysis. In some embodiments, the exosome membrane is not labeled, and exosomes are imaged directly in dark field to localize exosomes.

In some embodiments of the present disclosure, signal detection is further enhanced by binding multiple fluorescent nanoparticles to a targeted protein. In some embodiments, the secondary antibody is linked with multiple biotins to bind multiple fluorescent nanoparticles via streptavidin that is conjugated onto the fluorescent nanoparticle.

In some embodiments of the present disclosure, a single extracellular-vesicle dual imaging analysis (SEDIA) method is used to analyze the images. The method involves four major steps: 1) remove background noise of both mask (fluorescent or darkfield image) and target images using a selected baseline removal algorithm; 2) re-normalize all intensities in both images against a maximum value (the user have the options to set the maximum value in order to fine-tune the identification of algorithm based on the mask image, and the signals over the identified locations are obtained from the background-removed target image, 3) overlay two images to determine locations of exosomes, and 4) to extract target signals automatically to build a histogram. Using the single exosome analysis, small populations of cancer-derived exosomes in a vast background of non-cancer derived exosomes can be detected that would be undetectable by the bulk methods currently known in the art. The fraction of tumor-derived exosomes, which are very important in cancer diagnostics and monitoring, can be quantified using this method. Exosome subpopulations can be identified, and the exosome compositional heterogeneity can be discerned using the methods provided within. This information will be valuable for betting understanding tumor heterogeneity and help personalized treatment. Compared to the traditional Image J method, SEDIA is 20 times faster in terms of the overall time needed to process a pair of mask and target images. In other embodiments, a dual imaging single vesicle technology (DISVT) is used. In an exemplary embodiment, DISVT involves capturing extracellular vesicles (e.g., exosomes) from diluted biofluids onto a gold-coated multi-well chamber slide using an antibody anchored on the slide to bind to markers, e.g., CD81. Surface proteins of interests on the vesicles are labeled with antibody conjugated nanoparticles, e.g., gold nanoparticles (AuNP), and detected by dark field imaging. In some embodiments, the vesicles are pre-labeled with a lipophilic dye, e.g., CLS-PEG-Cy5), and detected by fluorescence imaging. The fluorescence image of vesicles and the dark field image of surface proteins bound with AuNPs can be overlayed to identify vesicles that are positive and/or negative for the surface proteins of interest. In some embodiments, the surface protein is a biomarker indicative of cancer.

### Extracellular Vesicle Sample Preparation

Extracellular vesicles such as exosomes may be found in any bodily fluid including, but not limited to, whole blood, plasma, ascites, breast milk, saliva, urine, sweat, semen, cerebrospinal fluid, and ocular fluid. In some embodiments, collected bodily fluids containing extracellular vesicles are processed to remove cells, debris, larger vesicles, and other matter that may confound detection of the extracellular vesicles. For example, samples may be filtered to remove the potentially confounding matter. In some embodiments, the samples may be centrifuged to remove the potentially confounding matter. In some embodiments, the sample comprising extracellular vesicles are tested without any pre-analysis processing. In some embodiments, the samples comprising extracellular vesicles are diluted prior to analysis. Diluting the samples may allow for sufficient sample volume for testing on a multi-well array.

Advantageously, the methods provided in the present disclosure only require submicroliter volumes of diluted plasma sample per marker. Over 200 samples can be processed on an array no bigger than a standard microscope slide, and the analysis can be completed in seconds. Therefore, compared to existing methods (including existing liquid biopsy protocols), the presently disclosed methods have several major advantages. The disclosed methods focus on single vesicle analysis but can also be used for bulk measurement via spectroscopic detection. The methods are highly sensitive, due to the use of fluorescent nanoparticles or metal nanoparticles that are orders of magnitude brighter than fluorescent dyes or non-metal nanoparticles. The methods are simple—capturing exosomes directly from plasma samples, labeling exosome membranes and the target protein with fluorescent probes that emit different visible signals and detecting these signals with a facile optical system. The disclosed methods are also very efficient, capable of processing over 200 samples on a single slide, conducting each measurement within seconds, and analyzing data with high automation. Lastly, the presently disclosed methods require very small amounts of samples (i.e., submicroliter of plasma samples (typically 100 times dilution for a plasma sample)).

### Arrays for Detecting and Characterizing Extracellular Vesicles

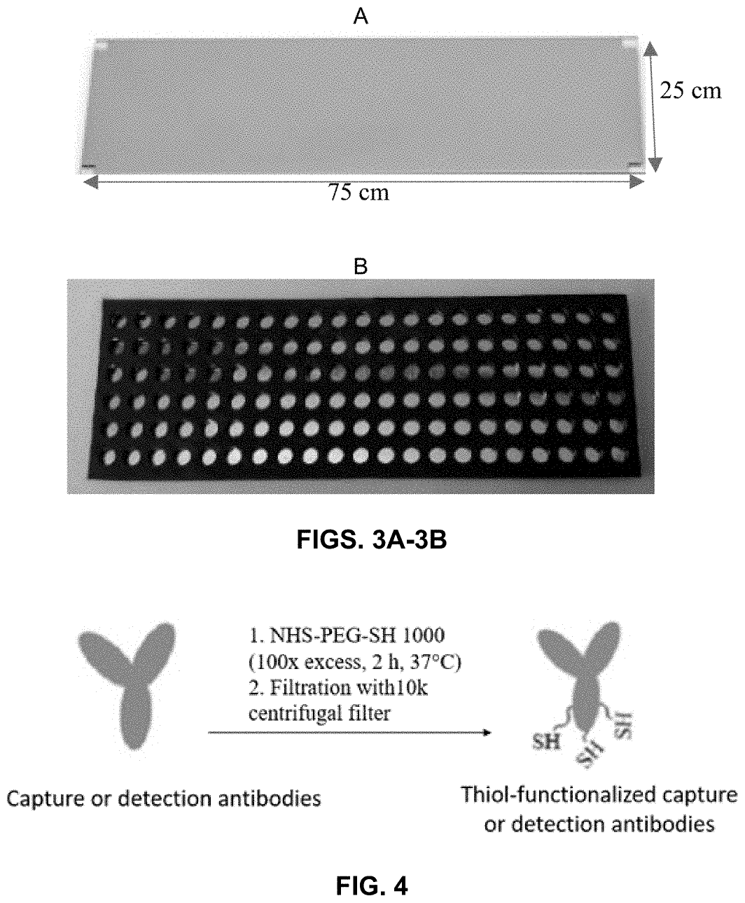

In some aspects of the present disclosure, a device is provided for the parallel processing and detection of extracellular vesicles in a plurality of samples. In some embodiments, the device comprises substrate having a functionalized surface to which capture antibodies are tethered. In some embodiments, the substrate is a glass slide coated with a substance that allows subsequent functionalization of the slide. For example, in some embodiments, the surface of the slide is coated with a gold film (FIG. 3A). The gold film can be between 1 and 200 nm thick. In some embodiments, the gold film is about 10 nm thick.

In some embodiments, the gold film is optically transparent. In some embodiments, the gold film is about 100 nm thick. A slide comprising a gold film surface can be fabricated using a magnetron sputtering technique that deposits a thin film of gold atoms onto a standard glass slide (i.e., 75 mm long×25 mm wide×1 mm thick). The gold surface of the slide may facilitate chemical modification of the slide's surface.

The slide may be functionally divided into two or more analytical zones where multiple samples can be analyzed in parallel. For example, the slide may be functionally divided into wells, and each well represents an analytical zone on the array. In some embodiments, the wells are formed by overlaying onto the slide a plastic (or other polymer) array having multiple holes. The array may be fabricated with a 3-D printer or any other available means (e.g., machined). Once overlaid, the holes on the array form the sample wells (FIG. 3B). To ensure tight fit of the array and the slide, pressure grease may be used to seal the array to the slide. In some embodiments, a gasket is situated between the slide and the array. In some embodiments, the slide may be functionally divided into two or more analytical zones based on the presence of distinct capture antibodies being tethered. For example, on one portion of the slide a first species of capture antibodies (e.g., antibodies that bind CD81) may be tethered. In a second portion of the slide, a second species of capture antibodies (e.g., antibodies that bind CD9) may be tethered. The second species of capture antibodies may bind to a surface protein of an extracellular vesicle that is different than the first species of capture antibodies. Accordingly, methods of the disclosure can be useful for multiplex analysis.

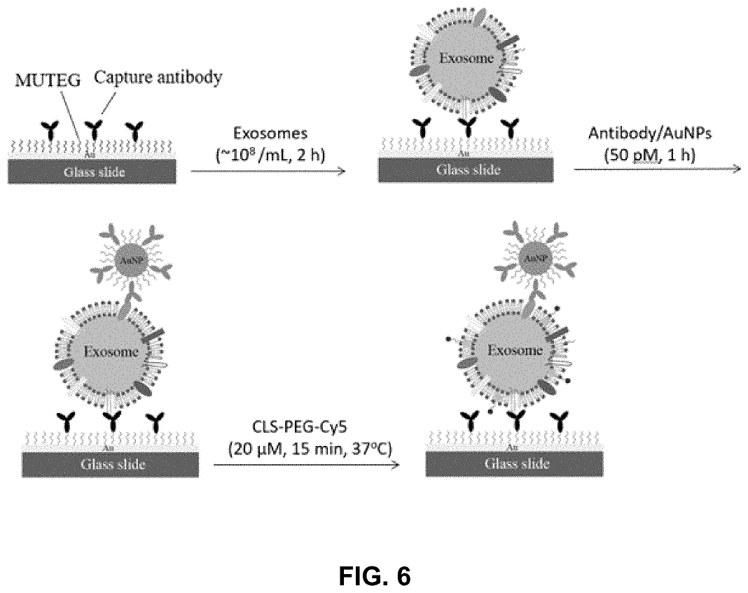

In some embodiments, the bottom surface of the well comprising the gold-coated film, is treated with a compound that reduces or eliminates nonspecific binding of the capture molecule. In some embodiments, the compound that reduces or eliminates nonspecific binding is 11-mercaptoundecyl tetra (ethylene glycol) (MU-TEG), or a similar compound (FIG. 6). Capture antibodies may be tethered to the substrate's functionalized surface. In some embodiments, the capture molecule is modified to facilitate binding to the functionalized surface of the substrate. For example, in some embodiments, the capture molecule is linked to a polyethylene glycol thiol (PEG-SH, molecular weight=1,000 Da) (FIG. 4). The specific capture of exosomes directly from blood with the capture molecules was confirmed with fluorescence imaging.

In other embodiments, extracellular vesicles are isolated or captured by using a lipophilic chemical layer on the surface of the array or substrate. Lipophilic molecules comprising an alkyl chain have high affinity for the lipid bilayer of molecules (i.e., extracellular vesicles) through hydrophobic interactions between the lipid membrane of the extracellular vesicle and the lipophilic molecules on the substrate. In some embodiments, the lipophilic molecule comprises a cholesterol group linked with CLS-PEG-SH. In some embodiments, the lipophilic molecule is 1,2-distearoyl-sn-glycerol-3-phosphoethanoloamine conjugated polyethylene glycol thiol (DSPE-PEG-SH; molecular weight=5,000 Da). The thiol group of the CLS-PEG-SH or DSPE-PEG-SH binds to the gold film on the surface of the slide, and the CLS or DSPE portion of the lipophilic molecule binds to the extracellular vesicle's membrane. Again, MU-TEG can be used to saturate the gold film surface of the array to reduce or eliminate nonspecific binding.

Detection methods may include use of a biochip array. Biochip arrays useful in the invention include protein and polynucleotide arrays. One or more markers are captured on the biochip array and subjected to analysis to detect the level of the markers in a sample.

Markers may be captured with capture reagents immobilized to a solid support as described herein, such as a biochip, a multi-well microtiter plate, a resin, or a nitrocellulose membrane that is subsequently probed for the presence or level of a marker. For example, a sample containing exosomes may be used to contact the active surface of a biochip for a sufficient time to allow binding. Unbound molecules are washed from the surface using a suitable eluant, such as phosphate buffered saline. More stringent eluants remove proteins that are not tightly bound.

Upon capture on a biochip, analytes can be detected by the Raman-based spectroscopy methods as described herein. In some embodiments, the analytes can be detected by additional methods as well. In one embodiment, optical methods, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry) are used. Optical methods include microscopy (both confocal and non-confocal), imaging methods and non-imaging methods. Immunoassays in various formats (e.g., ELISA) are popular methods for detection of analytes captured on a solid phase. Electrochemical methods include voltammetry and amperometry methods. Radio frequency methods include multipolar resonance spectroscopy.

Antibodies that specifically bind polypeptides and nucleic acid molecules present in extracellular vesicles may be used as hybridizable array elements in a microarray. The array elements are organized in an ordered fashion such that each element is present at a specified location on a substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3.i-e3.vii, 2000), MacBeath et al., (Science 289:1760-1763, 2000), Zhu et al. (Nature Genet. 26:283-289), and in U.S. Pat. No. 6,436,665, hereby incorporated by reference.

### Protein Microarrays

Proteins may be analyzed using protein microarrays. Such arrays are useful in high-throughput low-cost screens to identify alterations in the expression or post-translation modification of a polypeptide of the invention, or a fragment thereof. In one embodiment, a protein microarray as contemplated herein binds a marker present in a subject sample. Alterations in the level of the marker can be detected. In some embodiments, the protein microarray features a capture agent, such as an antibody or fragment thereof, bound to a solid support. Suitable solid supports include membranes (e.g., membranes composed of nitrocellulose, paper, or other material), polymer-based films (e.g., polystyrene), beads, or glass slides. The surfaces of these solid supports may be functionalized as described herein. For some applications, capture agents (e.g., antibodies that bind a marker of the invention) are spotted on a substrate using any convenient method known to the skilled artisan (e.g., by hand or by inkjet printer). The capture agents specifically bind to extracellular vesicles.

After the array is contacted with samples comprising extracellular vesicles, the protein microarray is hybridized with a detectable probe. Such probes can be polypeptide, nucleic acid molecules, antibodies, or small molecules labeled with a quantum dot. Probes can include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions, and such conditions are known to the skilled artisan and are described, for example, in Harlow, E. and Lane, D., Using Antibodies: A Laboratory Manual. 1998, New York: Cold Spring Harbor Laboratories. Unbound probes are removed and specifically bound probes are detected, for example, by fluorescence, enzyme activity (e.g., an enzyme-linked calorimetric assay), direct immunoassay, radiometric assay, or any other suitable detectable method known to the skilled artisan.

### Quantum Dot Probes for the Detection of Extracellular Vesicles

The present disclosure also provides methods of detecting and characterizing extracellular vesicles by using high throughput array and quantum dot (QD) probes in combination with metal particles. QD probes comprise a molecule having an affinity for a molecule of interest and a quantum dot. QDs have broad excitation wavelength wave compared to traditional fluorescent dyes yet have a narrow emission wavelength and a large Stokes shift. Due to these properties, a single excitation source (i.e., a laser) can elicit strong signals from different QDs. In comparison, a single excitation source used with traditional fluorescent dyes may not sufficiently excite some dyes that have excitation spectra that do not include the excitation source's wavelength. Because only a single excitation source is required, the methods and compositions described herein provide a less expensive, more efficient means of detecting extracellular vesicles.

Examples of QDs include QD525, QD565, QD605, QD655, QD705, and QD800 (Invitrogen). QD525 has an emission maximum of about 525 nm (i.e., a visible green signal) and can be sufficiently excited at a wavelength about 400 nm to about 488 nm. QD565 has an emission maximum of about 565 nm and can be sufficiently excited by a wavelength between about 350 nm and about 525 nm. QD605 has an emission maximum of about 605 nm and can be sufficiently excited with a wavelength between about 350 nm and about 600 nm. QD655 has an emission maximum of about 655 nm (i.e., a visible red signal) that can be sufficiently excited with a wavelength between about 350 nm and about 615 nm. QD705 has an emission maximum of about 705 nm and can be excited with a wavelength about 350 nm to about 630 nm. QD800 has an emission maximum of about 800 nm and can be excited with a wavelength about 350 nm to about 630 nm. Each QD can be excited using a laser that emits a wavelength sufficient to elicit a significant range (about 525 nm to about 800 nm) of possible emission maximums. In some embodiments, the laser is tuned to emit a 400 nm wavelength. In some embodiments, the laser is a 405 nm laser, 488 nm laser or other monochromatic commercially available laser with a wavelength from 400 to 500 nm. For example, because the emission maximums of the QD525 and the QD655 are distinct, both probes can be used to simultaneously detect two different molecules in a sample. For example, green QD525 can be used to detect a protein or other molecule that is only present in the lumen of an extracellular vesicle, while red QD655 can be used to detect a protein or other molecule that is only present on the surface of an extracellular vesicle. This is also true for all combinations of the QDs. The QDs can be used in combination with metal particles to allow for multiplex analysis.

### Quantum Dot Labeling of Extracellular Vesicles

According to some embodiments, methods disclosed herein contemplate detecting extracellular vesicles by labeling the vesicles with a QD probe in combination with a metal particle. In some embodiments, an antibody having an affinity for an epitope on an extracellular vesicle is conjugated to a QD. By incubating a sample comprising extracellular vesicles with the antibody conjugated to a QD, the antibody will bind to the epitope, thereby detectably labeling the extracellular vesicle.

Other methods of labeling extracellular vesicles are contemplated herein. For example, in some embodiments, extracellular vesicles, such as exosomes, are tagged with a composition that facilitates downstream labeling. In some embodiments, the tag comprises a lipid moiety that can be integrated into an extracellular vesicle's membrane. In some embodiments, the tag also comprises a moiety that can be recognized and bound by a composition comprising a detectable label. In some embodiments, the tag is a cholesterol (CLS)-polyethylene glycol-biotin (CLS-PEG-biotin) tag. Samples containing extracellular vesicles can be incubated with CLS-PEG-biotin tags, wherein the cholesterol moiety of the CLS-PEG-biotin tag becomes integrated into the extracellular vesicle's membrane.

The samples comprising tagged extracellular vesicles can be detectably labeled. Detectable labels, as contemplated herein, comprise a moiety that can recognize and bind to the extracellular portion of the tag that is integrated into an extracellular vesicle's membrane. For example, if the tag comprises a biotin moiety, then the detectable label may comprise streptavidin, which binds to biotin. The detectable label also comprises a quantum dot probe. For example, the detectable label may be QD655-labeled secondary antibody or streptavidin. In some embodiments, the sample comprising an extracellular vesicle having a CLS-PEG-biotin tag is incubated with a quantum dot-labeled streptavidin that recognizes and binds to the biotin moiety of the CLS-PEG-biotin tag. In some embodiments, the QD-labeled streptavidin allows detection of individual extracellular vesicles. In some embodiments, the quantum dot-labeled streptavidin allows detection of sample wells on an array comprising a sample having at least one extracellular vesicle.

In some embodiments, the slide wells are subsequently incubated with a primary antibody having an affinity for an epitope of a molecule on the surface of the extracellular vesicle, a molecule in the lumen of the extracellular vesicle, or both. In some embodiments, the molecule on the surface of the extracellular vesicle and the molecule in the lumen of the extracellular vesicle are the same molecule or related molecules. In some embodiments, the molecule on the surface of the extracellular vesicle is different than the molecule in the lumen of the extracellular vesicle. In some embodiments, the molecule on the surface of the extracellular vesicle, in the lumen of the extracellular vesicle, or both is a marker that is associated with an extracellular vesicle, thereby allowing positive identification of a sample well comprising an extracellular vesicle. In other embodiments, the molecule on the surface of the extracellular vesicle, in the lumen of the extracellular vesicle, or both is associated with a disease or condition or is indicative of from what cells or tissues the extracellular vesicle is derived.

Referring to the approach described in FIG. 6, one embodiment of the methods for detecting an exosome provides capturing and labeling a surface bound molecule as well a particular molecule within the lumen of the extracellular vesicle. A capture molecule having a polyethylene glycol thiol (PEG-SH) linker is attached to the Au-coated slide by forming an Au—S bond. The Au-coated glass slide comprising the linked capture antibodies is then treated with MU-TEG to reduce or eliminate nonspecific binding to the surface of the slide. The slide is then exposed to a sample comprising extracellular vesicles. In this figure, the extracellular vesicle comprises a targeted surface marker. Molecules present on the surface of the extracellular vesicle are recognized and bound by the capture molecule, thereby immobilizing the extracellular vesicle on the array.

The array is next contacted with metal nanoparticle linked to an antibody or antibody fragment comprising at least one constant region or at least one variable region, wherein the variable region has complementary determining regions (CDRs) that can recognize and bind to a surface bound molecule on the extracellular vesicle. The antibody may be polyclonal or monoclonal. Additionally, the antibody may be an isolated, naturally occurring antibody. Conversely, the antibody may be engineered. The surface bound molecule (i.e., biomarker) for which the antibody has an affinity may be a protein, lipid, polysaccharide or other carbohydrate, or any other molecule residing on or in the extracellular vesicle's membrane. The surface bound molecule may be associated with a specific type of cell, which would allow one skilled in the art to determine from what type of cell the extracellular vesicle is derived. In some instances, the surface bound molecule may be associated with health status. For example, a vesicle having a surface bound molecule that is a cancer antigen would indicate that the cell from which the vesicle derived is cancerous. Conversely, some surface bound molecules may be associated with a normal state. In still other embodiments, the absence of a particular surface bound molecule may be indicative of a disease state. And in some embodiments, the absence of a surface bound molecule may be indicative of the absence of circulating tumor cells (or at least a concentration of circulating tumor cells that is so low as to avoid detection). In still other embodiments, the absence of a surface bound molecule that is associated with a normal physiological state may indicate a genetic or acquired condition that prevents or reduces the expression of the surface bound molecule.

In some embodiments, the antibody that recognizes and binds to the surface bound molecule is linked to a AuNP.

In some embodiments, luminal molecules can also be assessed using the methods disclosed herein. As with labeling surface bound molecules, lumenal molecules may be recognized and bound by an antibody having an affinity for the molecule. In some embodiments, the antibody is linked with a metal nanoparticle. In some embodiments, the antibody is linked with a quantum dot probe. In some embodiments, the antibody is not labeled with a detectable probe.

In some embodiments, a sample is interrogated for the presence, absence, or amount of both surface bound molecules and lumenal molecules. In some embodiments, the surface bound molecule and the lumenal molecule are different. For example, in some embodiments, the surface bound molecule is a protein, while the lumenal molecule is a nucleic acid. In some embodiments, the surface bound molecule and the lumenal molecule are the same type of molecule, but are distinct species. For example, in some embodiments, the surface bound molecule and the lumenal molecule are both proteins, but the surface bound protein is a receptor, while the lumenal protein is a structural protein associated with the cell from which the vesicle is derived. In embodiments in which surface bound molecule and the lumenal molecule are different, the quantum dot probe for detecting the surface bound molecule may be different from the probe that is used to detect the lumenal molecule. In some embodiments, wherein the surface bound protein and the lumenal protein are the same, the quantum dot probes used in their detection may be the same as well. Additionally, the methods and compositions comprised herein also contemplate the simultaneous detection of multiple lumenal and/or surface bound molecules. Some embodiments of the present disclosure provide for multiple distinct quantum dot-labeled antibodies, each recognizing and binding to a different surface bound or lumenal molecule or a different primary antibody that is bound to a different surface bound or lumenal molecule. The system can be effectively tuned as described above.

### Detecting Quantum Dot-Labeled Extracellular Vesicles and Molecules

Detection of QD-labeled extracellular vesicles and molecules requires an excitation source and an emission collector. In some embodiments, the excitation source used to excite the QD probes is a laser. Because QDs have broad excitation spectra, the excitation source can be a single laser rather than a matched-laser system, wherein separate lasers with different wavelengths are required to efficiently excite the probes. In detection schemes using traditional fluorescent dyes, a single laser may be able to elicit emissions from two different dyes, but at least one of the dyes will have a reduced emission because the laser wavelength is not near the excitation wavelength maximum of the fluorescent dye. Reducing the componentry of the detection apparatus, reduces the cost of the machinery necessary to perform the methods described herein.

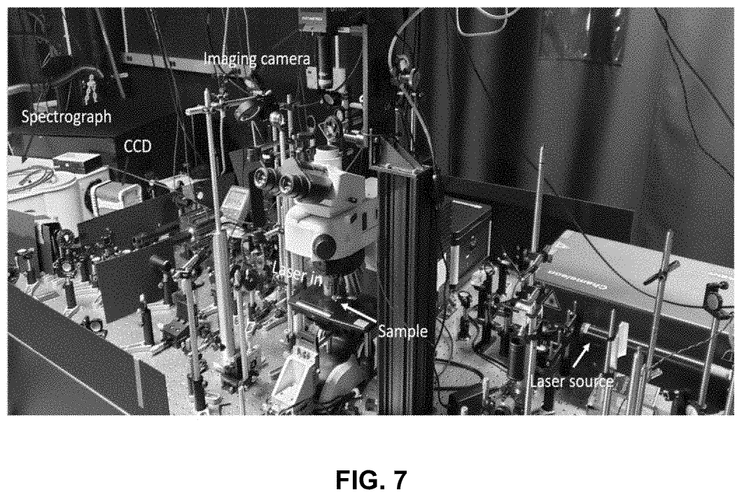

Commercially available or custom-built confocal fluorescence microscope systems may be used to detect labeled extracellular vesicles. Referring to FIG. 7, which depicts a custom multifunctional optical microscope system, a laser is used to excite a sample on a 3D stage. In some embodiments, the laser is a tunable laser. In some embodiments, the laser is configured to emit light onto the 3D stage at an acute angle with respect to the stage. Reflected fluorescent signal from the sample is collected by the objective and, after passing through the beam splitters, is filtered by an appropriate long-pass filter (to block the laser excitation) and refocused onto an intermediate image plane where a small pinhole is used to select single extracellular vesicles of interest. A series of lenses filters process the emitted signals allowing both nano-imaging of the fluorescing extracellular vesicles as well as capture by a spectrometer. A nano-image (typically captured by a charged couple device (CCD)) of fluorescing extracellular vesicles provides a qualitative visual image of the single vesicles present in a sample. In some embodiments, the spectrometer data provides a quantitative characterization of the detected extracellular vesicles (i.e., bulk analysis of the vesicles in the sample). The spectrometer and CCD camera are optimized for the visible frequency with up to 95% quantum efficiency that is ideal for single extracellular vesicle measurements.

### Magdye Labeling and Detection of Extracellular Vesicles

Fluorescent nanoparticles other than QDs can also be used to detect the low abundance of exosome surface proteins. One example is Magdye that comprises magnetic nanoparticles (10 to 100 nm) coated with multiple fluorescent dyes such as Cy5 (commercially available at Ocean Nanotech, LLC). The Magdye can be 300 times brighter than the dye itself due to the large number of dyes coated on the nanoparticles. The exosome labeling method using Magdye is the same as described as QDs above. For mask imaging, exosomes can be directly imaged in dark field without further labeling or imaged under fluorescence microscope after membrane labeling with a second dye with different color. An example is target imaging with Cy5 and membrane labeling with Alex Flor 405. The two dyes have separated emission properties and thus do not interfere each other. Any other dual-color dyes can also be used provided that their excitation and emission spectra do not overlap.

### Dual Imaging Single Vesicle Technology (DISVT)

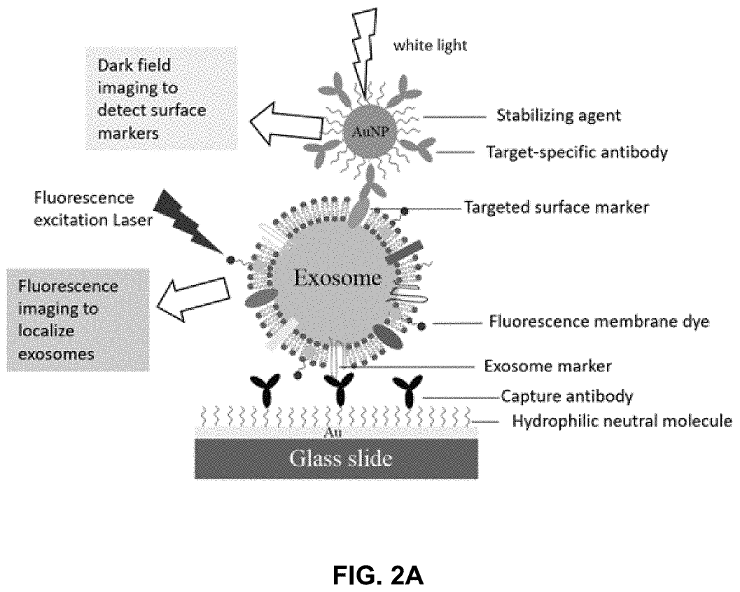

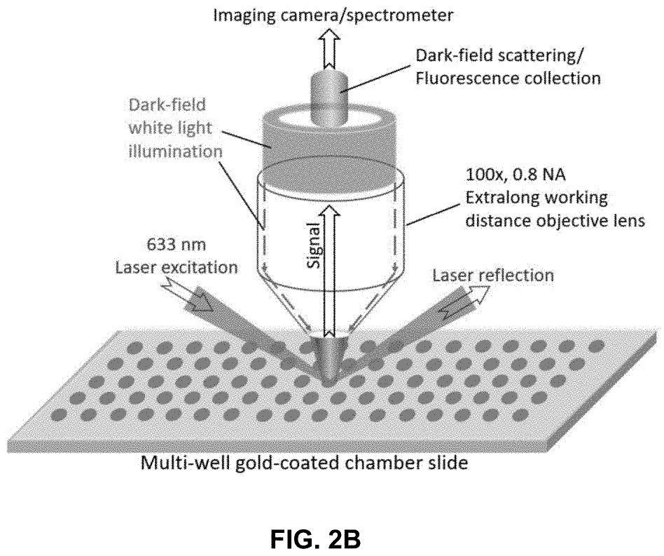

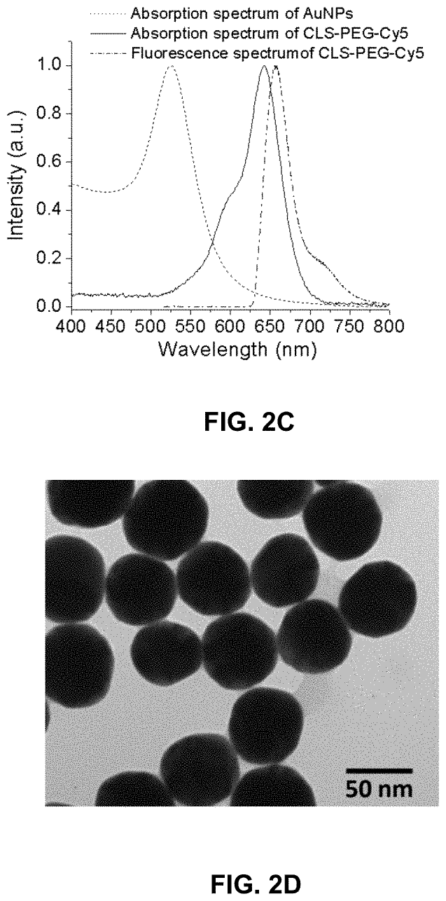

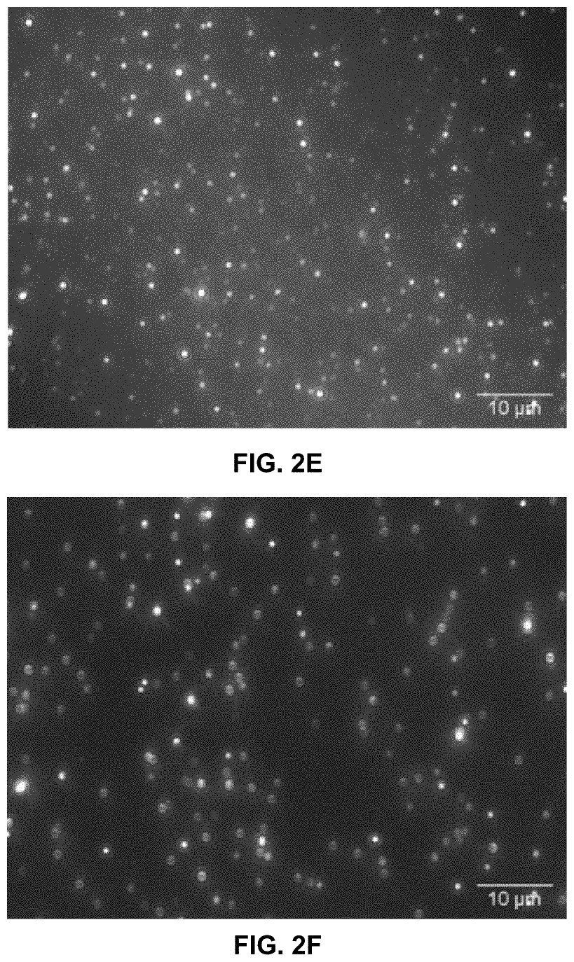

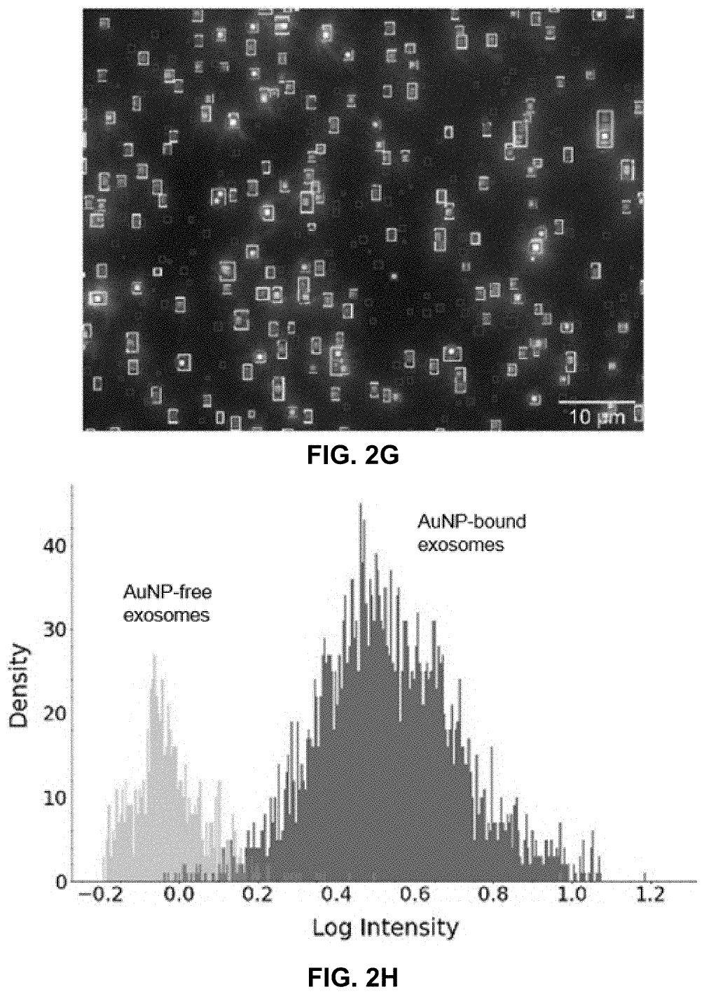

In one aspect, this disclosure provides a dual imaging single vesicle technology (DISVT) for detecting and characterizing extracellular vesicles, such as exosomes. FIGS. 2A-2H show an overview of DISVT. In an exemplary embodiment, the DISVT captures exosomes from diluted biofluids onto a gold-coated multi-well chamber slide using an antibody anchored on the slide to bind to exosome markers, such as CD81. Surface proteins of interests on exosomes are labeled with antibody conjugated nanoparticles, e.g. AuNP probes, and detected by dark field imaging. To localize exosomes on the Au slide, the exosomes are then labeled with a lipophilic dye, e.g., CLS-PEG-Cy5, and detected by fluorescence imaging, e.g., using a high sensitivity laser excited fluorescence imaging module (FIG. 2A and FIG. 2B). Due to the strong localized surface plasmon resonance (LSPR) (FIG. 2C), the AuNPs exhibit strong light scattering properties. Correspondingly, AuNPs can be detected at single particle sensitivity under dark field that detects scattered light from objects. This provides the ability to detect rare molecules of interests on the surface of small EVs such as exosomes with AuNPs. In some embodiments, the AuNPs are 60 nanometers in diameter and are homogenous in size (FIG. 2D), which allows for homogenous imaging. Without limiting the scope of the disclosure, in some embodiments exosomes are labeled with Cy5 because the emission peak of Cy5 has substantially zero to minimal overlapping with the LSPR band of the AuNPs (FIG. 2C). Thus, energy transfer between AuNPs and the dye is negligible.

In some embodiments, fluorescence imaging is coupled to dark field imaging, allowing for simultaneous detections of exosomes via fluorescence imaging with fluorescent membrane dyes, e.g. CLS-PEG-Cy5, and their surface proteins via dark field imaging with light scattering nanoparticles, e.g. AuNPs, using a single optical microscope. Superimposing the fluorescence image (the mask, FIG. 2E) and a dark field image (the target, FIG. 2F) of the labeled exosomes reveals the AuNP-bound and AuNP-free exosomes (FIG. 2G). Extracting the pixel intensity in the target image at the location of exosomes in the mask image gives the exosome population density histogram. This histogram informs the profile of the AuNP-bound exosomes and AuNP-free exosomes (FIG. 2H). Calibration with isotype IgG (the control AuNPs, to correct nonspecific binding of antibodies) gives the fraction of exosomes that are positive for the targeted protein marker, Fₚ, over the captured exosomes and the expression level of the targeted marker on total analyzed exosomes, ξₚ. The mean value of the Log Intensity of the histogram profile of the marker-negative exosomes is normalized to 1 to facilitate the comparison of different samples and repeated experiments at different days. Accordingly, compositions and methods described herein offer the capability to quantify percentages of tumor-derived extracellular vesicles from biofluids for disease diagnostics and monitoring.

In some embodiments, the dark field and fluorescence images are acquired with a customized dual imaging microscope in which fluorescence is excited by a light that is configured to strike a sample at an angle with respect to the objective lens (e.g., a 100× objective). For example, the light may be emitted by a light emitting device (e.g., a laser, such as a red diode laser or a mercury arc lamp) and onto a sample at an angle of between 35 and 55 degrees with respect a solid support (e.g., a slide) on which the sample is located during analysis. Advantageously, by emitting light at an angle with respect to the sample, a larger sample area can be illuminated and thus detected in a single image acquisition. In addition, instance of photobleaching is reduced. In some embodiments, the angle comprises an angle of 45 degrees. In some embodiments, the angle comprises an angle of greater than 45 degrees and less than 90 degrees. In some embodiments, the angle comprises an angle of less than 45 degrees. However, as a person skilled in the art will readily appreciate, any number of different configurations can be used according to the scope of this disclosure. For example, in some embodiments a 45× objective is used. In some embodiments, a 60× objective is used. In some embodiments, a green diode laser is used. In some embodiments, a mercury arc lamp is used.

In some embodiments, the dual images (the mask and target images) are analyzed with Image J (imagej.nih.gov/ij/features.html). In some embodiments, the mask-target images are analyzed using a Single Extracellular-vesicle Dual Imaging Analysis (SEDIA) method, a semi-automatic imaging analysis platform developed by the inventors using Bash scripts, Python, and ImageJ. This code analyzes multiple images simultaneously within seconds, which is ˜50 times faster than using ImageJ that analyzes one image at a time (5 min with SEDIA versus 4 h with Image J per sample), which dramatically enhances the efficiency of data analysis. In some embodiments, the mask-target images are analyzed using a fully automated Single Extracellular-vesicle Dual Imaging Analysis (autoSEDIA) method, which uses python coding only from image pre-processing to data output.

### Image Analysis

In one aspect, this disclosure provides an imaging analysis method for characterizing extracellular vesicles. The method can be used to analyze images generated by methods of the disclosure and provide biologically relevant data useful for informing on a state of condition of a subject from which extracellular vesicles were derived. In some embodiments, the imaging analysis is based on Python code. It can be used to count extracellular vesicles and provide measurements of the extracellular vesicle's marker expression. The method is sometimes referred to herein as SEDIA, standing for Single Extracellular-vesicle Dual Imaging Analysis, a semi-automatic method based on Bash scripts, Python, and ImageJ. In some embodiments, the program uses image pairs as input and returns the measured properties of each detected extracellular vesicle from the images. In some embodiments, the analysis follows the following procedure. The procedure is described herein.

First, Bash scripts are used to automatically import a set of raw fluorescence images (EV mask) and dark field images (protein target) into ImageJ. Then, Python scripts are used within ImageJ itself using the native Jython interpreter to carry out the automatic implementation of a custom image analysis process utilizing ImageJ algorithms. Each set of images are processed with a 2D implementation of a zeroth-order Savitsky-Golay smoothing filter to suppress pixel-to-pixel noise, followed by 5×5 custom convolution kernel to reduce unequal illumination. Next, Otsu's method is used to set a threshold for detecting spots for each image, then each image is converted to binary format.

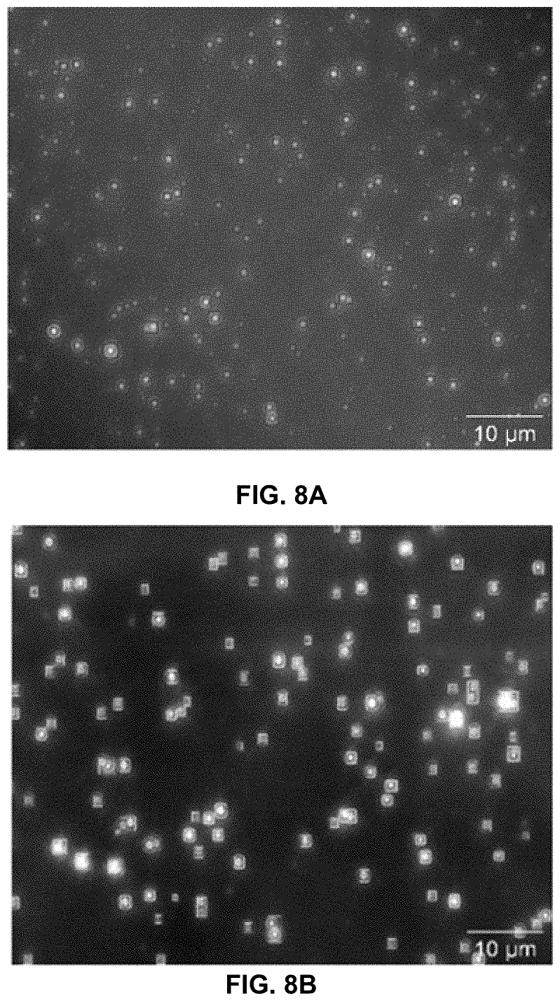

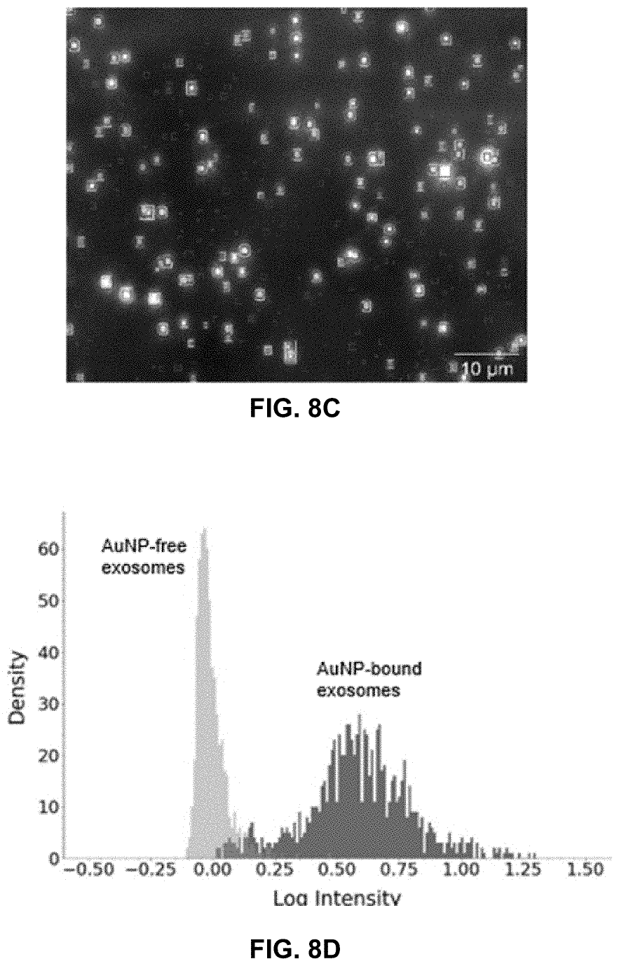

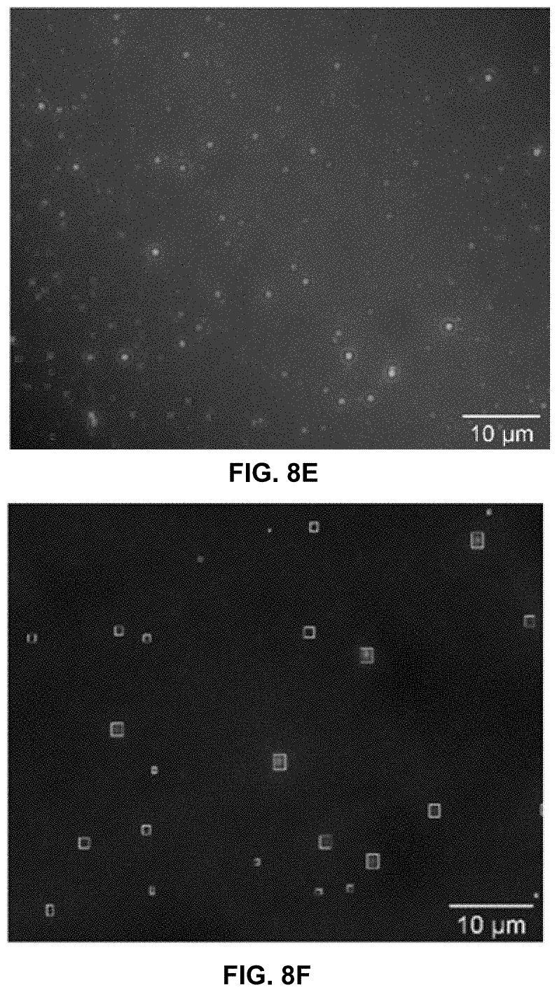

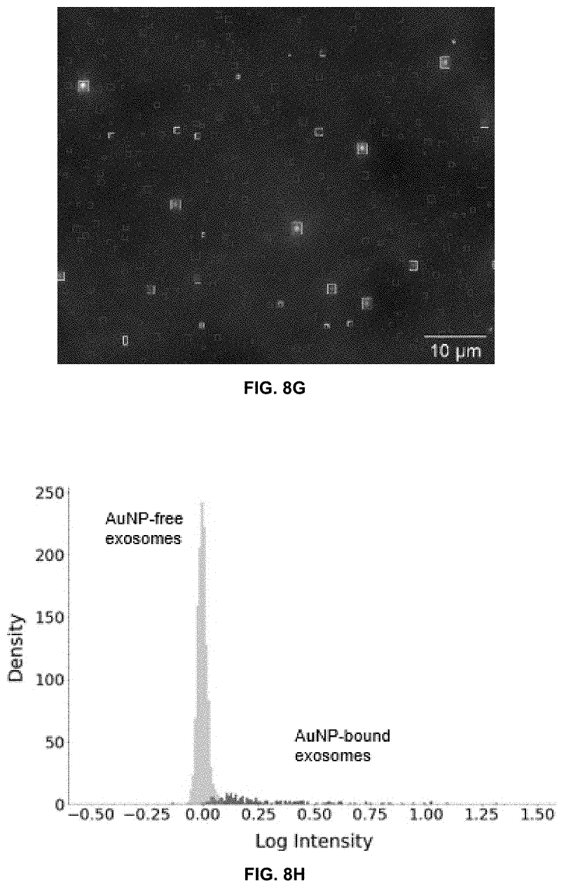

A custom Python script utilizing scikit-image is then used to label the dye-bound EVs in the mask image (FIG. 8A and FIG. 8E) and AuNP-bound EVs in the target image (FIG. 8B and FIG. 8F), followed by the superimposing of the two images with overlapping label areas of 50% or greater being accepted as marker positive EVs (FIG. 8C and FIG. 8G). The logarithm of the normalized integrated pixel intensity in unit area of labeled particles in the dark field image at the EV mask locations gives the population density histogram that includes two populations, AuNP-bound EVs and AuNP-free EVs (FIG. 8D and FIG. 8H). To compensate for the variation of instrument responses, the density profile is normalized by defining the maximum intensity of the AuNP-free EV band to be 1 (and thus its logarithm to be zero). The fraction of marker-positive EVs to total EVs, Fₚ, is then calculated by taking the difference of the fraction of Au-bound EVs using the antibody-conjugated AuNPs and the fraction of Au-bound EVs using the IgG-conjugated AuNPs. The difference of the average normalized intensity of all EVs between antibody-conjugated AuNPs and IgG-conjugated AuNPs is calculated as ξₚ to represent the expression level of the targeted protein on individual EVs. In some embodiments, the dual images can be analyzed with a fully automated SEDIA (autoSEDIA) method, which only uses python coding in the entire imaging analysis process, from image pre-processing to image labeling and to data extraction.

### Diagnosing and Treating Disease

The compositions and methods of the present disclosure may be used to diagnose disease. As described supra, molecules associated with disease can become integrated into or onto an extracellular vesicle. The detection and characterization of such vesicles and their disease-associated molecules can provide information relevant for diagnosing a disease, determining the progression or regression of disease, and treating disease. For example, some cancer cells express particular tumor antigens that are associated with a particular stage of the disease. A tumor antigen is a protein that is overexpressed in certain cancers (e.g., breast cancer) that can be used as marker, in some cancers, for determining patient prognosis. Samples obtained from a subject having or suspected of having breast cancer may be analyzed for the presence or absence of a tumor antigen. Those individuals having extracellular vesicles that are positive for the tumor antigen be diagnosed with cancer. In some cases, a subject having cancer who has tumor antigen-positive extracellular vesicles may have a poorer prognosis that a cancer patient without the tumor antigen-positive extracellular vesicles.

A subject having a disease may undergo periodic testing to determine if the level of a cancer marker is increasing, decreasing, or static. For example, a subject having tumor antigen-positive cancer may surveil the amount of tumor antigen-positive extracellular vesicles present in subsequent samples to quickly determine if there is any alteration in the amount of tumor antigen-positive vesicles. If the amount of tumor antigen-positive vesicles in a sample is greater than that observed in a previous sample, the subject's cancer is likely progressing or not responding effectively to treatment. If the number of tumor antigen-positive extracellular vesicles remains static relative to an earlier sample, the disease may be responding treatment sufficiently to stop disease progression, but perhaps not to a level sufficient for disease regression or remission. Conversely, if the number of tumor antigen-positive extracellular vesicles decreases relative to an earlier sample, the subject's disease may be regressing, and the absence of such vesicles may signify remission.

It is contemplated that the compositions and methods in the present disclosure can be used to identify the disease stage of a subject. For example, some markers associated with early stages of disease may be lost, or expressed at a reduced level, as the disease progresses. Thus, the marker profile of a subject's extracellular vesicles can be compared to a reference profile or profiles and a determination made regarding the particular stage of disease the subject is experiencing.

### Kits

The present disclosure contemplates kits for characterizing extracellular vesicles. The kits can comprise metal nanoparticles (e.g., AuNPs) for labeling biomarkers present on the surface of extracellular vesicles. In some embodiments, the biomarkers may be indicative of a disease such as cancer. In some embodiments, the metal nanoparticles are provided in a vessel comprising an aqueous solution. In some embodiments, the metal nanoparticles are pre-linked to antibodies or fragments of antibodies for binding to biomarkers present on the surface of extracellular vesicles. In other embodiments, the metal nanoparticles are provided with reagents useful for linking the metal nanoparticles with the antibodies or fragments thereof that are of interest. For example, in some embodiments, the kit includes antibodies in combination with PEG-SH. In some embodiments, the kit includes mPEG-SH 1000. In some embodiments, the kit comprises a tag primary antibody having an affinity for at least one surface bound molecule or at least one lumenal molecule; and a secondary antibody having an affinity for the primary antibody, wherein the secondary antibody comprises a quantum dot label. In some embodiments, the kit further comprises at least one additional primary antibody. In some embodiments of the kits that comprise more than one primary antibody, the kit also comprises at least one additional secondary antibody, wherein each secondary antibody is labeled with a different quantum dot. In some embodiments of the kit, the kit further comprises an array for detecting extracellular vesicles and directions for its use. In some embodiments, the kit further comprises instructions for printing on a 3D printer an array suitable for interrogating samples for the presence or absence of extracellular vesicles and/or molecules associated said extracellular vesicles. In some aspects, a kit comprises an array for detecting extracellular vesicles and directions for its use. In some embodiments, a kit comprises instructions for printing an array on a 3D printer that is suitable for interrogating samples for the presence or absence of extracellular vesicles and/or molecules associated said extracellular vesicles.

The following examples are put forth to provide those of ordinary skill in the art with a complete disclosure and description of how to make and use the assay, screening, and therapeutic methods of the invention, and are not intended to limit the scope of what the inventors regard as their invention.

In particular, the examples below describe technologies for accurate and sensitive diagnostics and monitoring are desired to combat cancer. Traditional tissue biopsy is invasive, costly, impractical for repeated testing, and unattainable for some cancer types. In addition, tumors are heterogeneous and evolve over time. Thus, biopsy data collected from the limited times can be biased and can mislead clinical decisions. Additionally, the primary tumor is not accessible after surgery, inhibiting treatment monitoring. These limitations could be addressed by probing biomarkers from primary and secondary tumors with noninvasive liquid biopsy techniques. Bulk methods are inherently contaminated with normal exosomes and they require large amount of samples. Single exosome profiling of surface proteins can probe tumor-derived exosomes in the presence of abundant non-tumor exosomes, providing sensitive, precise, and quantitative information superior to bulk methods. It would provide unprecedented insight into biological events and invaluable information for new biomarker discovery. However, single exosome surface protein profiling requires high sensitivity and high resolution technologies because of the low abundance (down to single digit) of antigens on the small exosomes. In addition, the technologies need to meet clinical criteria including easy-to-use, fast turnaround, and low cost so that they can benefit broad range of customers. The dual imaging single vesicle technology (DISVT) addresses the technical need using unique capabilities of nanotechnology and optical imaging to analyze surface protein markers on individual exosomes in body fluids. The DI-SVT combines high sensitivity gold nanoparticles (AuNPs) and optical imaging, facile direct exosome capture, and fast mask/target imaging to overcome the technical barriers in molecular analysis of exosomal surface markers for clinical applications and basic research.

### EXAMPLES

### Example 1: Preparation of Au Chamber Slide

Exosomes were captured and analyzed on gold (Au)-coated standard microscope glass slide (75×25×1 mm) (FIG. 3A). The Au film, which was 100 nm in thickness, was used to facilitate surface modification. The Au slide was prepared from Au-coated silica chip that is commercially available (Angstrom Engineering, LLC) by a stripping method with epoxy. Briefly, prepare an epoxy solution was prepared by adding Part A: Part B epoxy with 1:1 ratio and mixing for 2 hours on a shaker. The epoxy mixture was added to Au wafter (100 nm Au thickness) (5 microliters for wash 0.5×0.5 inch), then a glass slide was placed onto the Au wafer. Heating the Au wafter at 150 degrees Celsius for 2 hours. After the epoxy had set, the Au wafter was cooled to room temperature (RT) and then the Au coated glass slide was removed. The Au slide was then separated into multiple wells using a 3D printed cassette with tunable well size and inter-well distance (FIG. 3B). In the example shown in FIGS. 3A-3B, the diameter, spacing, and height of each well was 2.5, 1.0, and 1.0 mm, respectively. The maximal capability of each well was 4.9 microliters. The cassette was removed, washed, and reused without damage. It is fabricated with a Formlabs Form 2 3D printer.

### Example 2. Functionalization of Antibody with Thiol Group

Capture or detection antibodies were functionalized with a thiol using a NHS-polyethylene glycolthiol linker (NHS-PEG-SH, molecular weight 1000) before use to functionalize the Au chamber slide or make AuNP probe (FIG. 4). This was done by reacting the antibody (10 microgram) with 100 times molar excess of NHS-PEG-SH at 37 degrees Celsius for 2 hours. The free NHS-PEG-SH was separated by filtration with a 10K centrifugal filter. The antibody-PEG-SH was stored at 4 degrees Celsius before use.

### Example 3. Functionalization of the Au Chamber Slide with Capture Antibodies

To functionalize the Au chamber slide to molecularly capture exosomes, the Au chamber slide was immersed in a phosphate buffer solution (PBS) containing 4.5% bovine serum albumin (BSA) and 25 microgram per milliliter CD81 antibody linked with PEG thiol (molecular weight 1000) for overnight at room temperature (RT). Then, the slide was washed with PBS containing 0.01% tween 20 (PBST) three times. The CD81 antibody-bound slide was then incubated with 0.1 millimolar 11-mercaptoundecyl tetra (ethylene glycol) (MU-TEG) for 30 min to saturate the surface to eliminate nonspecific bindings.

### Example 4. Preparation of Antibody-Conjugated AuNPs

To conjugate the metal nanoparticles, 60 nanometer AuNPs were purchased from Ted Pella, Inc. As described herein (FIG. 5), 200 microliter of AuNPs were mixed with antibody-PEG-SH 1000 (10,000 molar excess to AuNPs) for overnight (about 16 h) at 4 degrees Celsius. Then, methoxy-PEG-SH 5000 (30,000 molar excess to AuNPs) was added and stirred for 1.5 h at RT. The antibody-conjugated AuNPs were purified by three rounds of centrifugation (10,000 revolutions per minute, 10 minute) and stored in PBS at 40 degrees Celsius in the presence of 0.05% sodium azide.

### Example 5. Exosome Capture and Labeling

FIG. 6 shows a schematic of the methodology for exosome capture and labeling that was used. As illustrated, three major steps were performed: 1) Exosome capture: in this step, exosomes in cell culture media or diluted plasma about 10⁸ per milliliter) and filtered with 0.2 micrometer polyethersulfone (PES) filter to get rid of cell debris and larger other types of extracellular vesicles. 2) Protein labeling with AuNP probes: this is done by incubating exosomes with 50 picomolar antibody conjugated AuNPs in the presence of 5% BSA for 1 hour at RT. 3) Exosome membrane labeling with fluorescence probes: this is done by incubating the exosomes with 20 micromolar cholesterol-PEG-Cy5 (CLS-PEG-Cy5, PEG molecular weight 2000) for 15 minutes at 37 degrees Celsius. Exosomes were washed between each step with PBST for 3 times and then PBS for 3 times.

### Example 6. Dual Fluorescence and Dark Field Microscopic Imaging System

An optical system for dual imaging using was constructed by customizing a Nikon LV 150N microscope (FIG. 7). The system allows for bright/dark field imaging with a halogen illumination lamp. Fluorescence imaging was accomplished with an excitation laser in an angled direction (˜45 degree relative to the sample surface) from the side of the 100× objective lens. The angled illumination allows for high resolution fluorescence imaging on micrometer scale without sacrificing spatial resolution. A Griot continuous wave He laser (wavelength at 632.8 nanometer) was used to excite the fluorescence signals of Cy5. The optical system enabled uniform images of the sample with high resolution and high sensitivity without an expensive and complicated commercial confocal microscope. This facile imaging systems allowed complete image acquisition within seconds per sample.

### Example 7. Single Extracellular-Vesicle Dual Image Analysis (SEDIA)

A semi-automated imaging analysis method, Single Extracellular-vesicle Dual Image Analysis (SEDIA) has been developed using Bash scripts, Python, and ImageJ. This method analyzes multiple images simultaneously within seconds, which is ˜50 times faster than using ImageJ alone which analyzes one image at a time (5 min with SEDIA versus 4 h with Image J per sample). The SEDIA reads a pair of the raw fluorescence image (EV mask) and dark field image (protein target). Both images are pre-processed, including Savitsky-Golay smoothing, kernel convolution, and background correction with the Otsu threshold. Then, the python script labels EVs on the processed mask image and AuNPs on the target image, followed by superimposing the two images to identify the AuNP-bound EVs and AuNP-free EVs. FIGS. 8A-8E show the labeling, superimposing, and signal extraction during image analysis with SEDIA using plasma EVs from a stage III HER2-positive breast cancer patient as an example. FIG. 8A and FIG. 8E show the fluorescence image of the plasma EVs labeled with HER2/AuNPs and IgG/AuNPs, respectively. FIG. 8B and FIG. 8C show the dark field image of the plasma EVs labeled with HER2/AuNPs and IgG/AuNPs, respectively. FIG. 8C and FIG. 8G show the superimposed image of the plasma EVs labeled with HER2/AuNPs and IgG/AuNPs, respectively. The superimposed images show the AuNP-bound exosomes (white boxes) and AuNP-free exosomes (grey boxes).

The logarithm of the pixel intensity of the dark field image at the locations of EVs identified on the fluorescence mask image gave the population density histogram that reflected the marker expression profile on individual EVs. Two populations of EVs were shown on the population density histograms, AuNP-bound EVs (dark grey peaks in FIG. 8D and FIG. 8H) and AuNP-free EVs (light grey peaks in FIG. 8D and FIG. 8H) for this HER2-positive EV sample. To facilitate the comparison of the experiment results from different days and different samples, the dark-field image intensity of the AuNP-free EVs was normalized by their average values. As a result, the histogram of all marker-negative EVs was centered at zero on the x-axis.

The fraction of marker-positive EVs over all analyzed EVs, Fₚ (p is the name of the targeted surface protein), was the difference of calibrated fraction of AuNP-bound EVs between antibody-conjugated AuNPs and the IgG-conjugated AuNPs. In the example shown in FIG. 8, FHER2 was determined to be 0.28, suggesting that 28% of CD81-positive plasma EVs were HER2-positive for this patient. The mean expression level of the protein per EV over all examined EVs, ξₚ can be calculated based on the normalized pixel intensity with calibration by IgG control. This parameter represents an ensemble property and is thus comparable to protein expression levels measured by bulk methods such as ELISA.

### Example 8. Early Detection of Breast Cancer Using DISVT

To test the clinical potential of DISVT for cancer detection, proof-of-concept studies were conducted using HER2-positive breast cancer as the disease model. HER2-positive breast cancer is one of the major breast cancer types, account for 20-30% of all cases. Plasma samples were purchased from BioIVT from ten early-stage (n=8 for stage I and n=2 for stage II) HER2-positive breast cancer patients, ten stage III HER2-positive breast cancer patients, ten stage III HER2-negative breast cancer patients, and ten healthy donors. They were diluted with PBS to an EV concentration around 10⁸ per milliliter. The diluted plasma was filtered with 0.2 micrometer PES filter to separated cell debris, protein and EV aggregates, and extracellular vesicles that were larger than 200 nanometer such as microvesicles to obtain exosome-like EVs. Using the procedures described above, the exosome-like EVs from each human subject were captured and labeled targeting HER2 marker with isotype IgG as the control. Fluorescence and dark field images were collected using the constructed dual imaging microscope system with fluorescence excitation laser at 45 degree relative to the sample plane. Images were analyzed with SEDIA to derive the EV population density histograms and to calculate FHER2 for each subject.

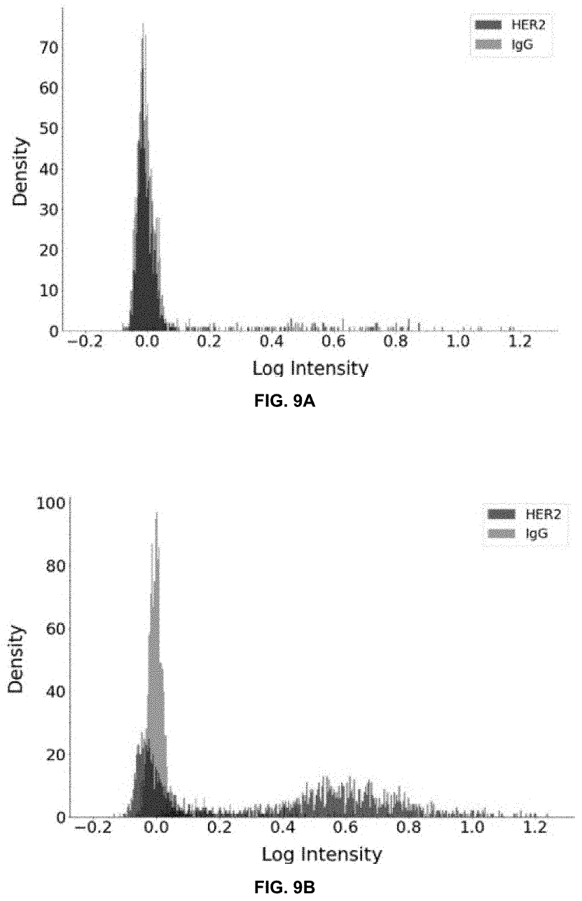

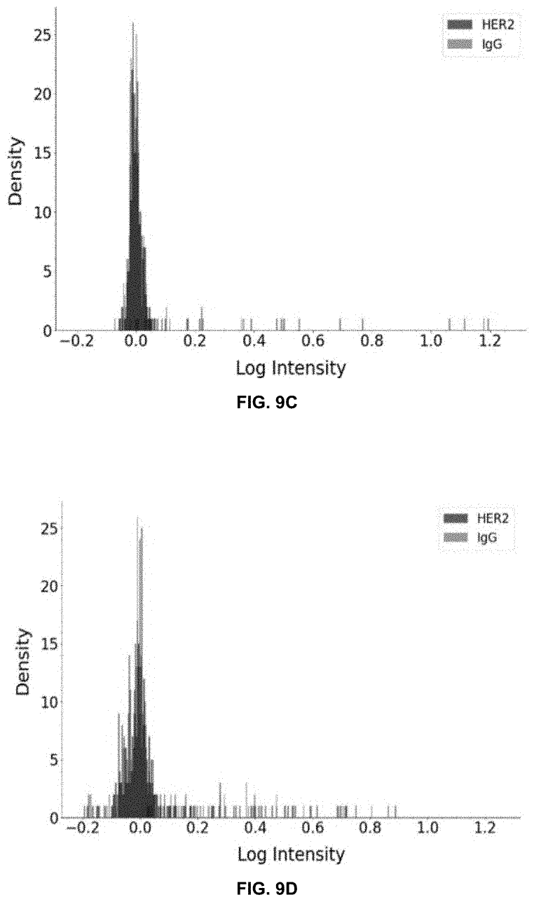

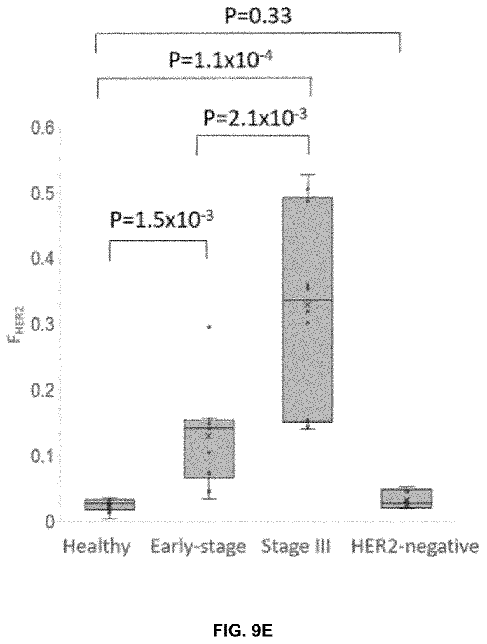

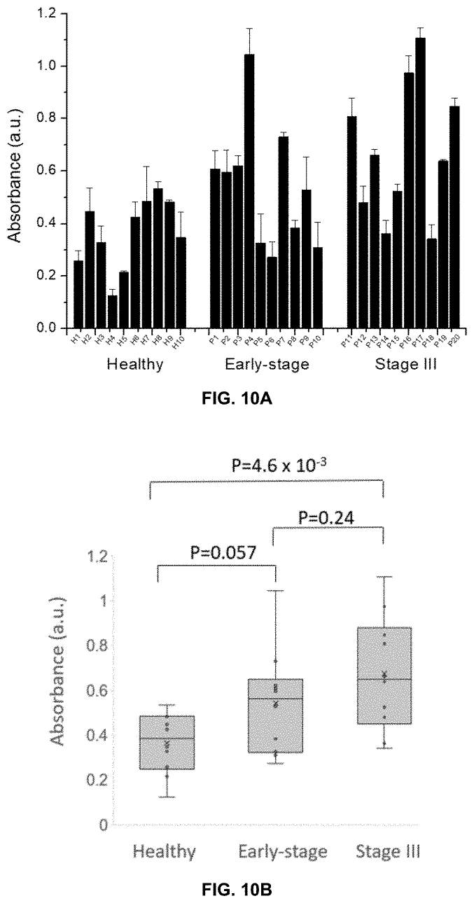

FIGS. 9A-9D show an example of the EV population density histogram for each group of human subjects, labeled with HER2/AuNPs (dark grey) and IgG/AuNPs (light grey). The results showed that the stage III patient showed more HER2-positive EVs than stage I. The two controls, HER2-negative stage III breast cancer patients and healthy donors, did not show much difference between their HER2/AuNPs and IgG/AuNPs. FIG. 9E shows a comparison of FHER2 values among the four groups. The FHER2 values for the healthy and HER2-negative control groups were lower than 3%. The FHER2 values for the patients exhibit large heterogeneity, ranging from 3% to 17% for early-stage patients and 4% to 50% for stage III patients. The early-stage patients had an average of 9.5% HER2-positive EVs and the stage III patients had an average of 32.9% HER2-positive EVs.

The data were statistically analyzed with analysis of variance (ANOVA) with p≤0.05 being significantly different. The results showed that the FHER2 of the early-stage patients and stage III patients were both significantly different from that of the healthy control, with p=1.5×10⁻³ for early-stage patients and p=1.1×10⁻⁴ for stage III patients. The FHER2 of early-stage patients was also significantly different from stage III patients (p=2.1×10⁻³). This suggests that our DISVT can detect cancer at both early-stage and locally-advanced stage (stage III) by quantifying the fraction of cancer marker-positive EVs of the CD81-positive subtype. It can also differentiate early-stage from locally-advanced stage. There was no significant difference between the healthy control and HER2-negative stage III patients (p=0.33), indicating high specificity of DISVT for detection of targeted cancer markers on plasma EVs.

For comparison, we analyzed the HER2 expression of the same subjects using the sandwich ELISA. Similar to DISTV, we used CD81 to capture EVs in the ELISA analysis. In contrast, significant difference was only observed between the healthy control and stage III patients (p=4.6×10⁻³) for the ELISA method (FIGS. 8A-8B).

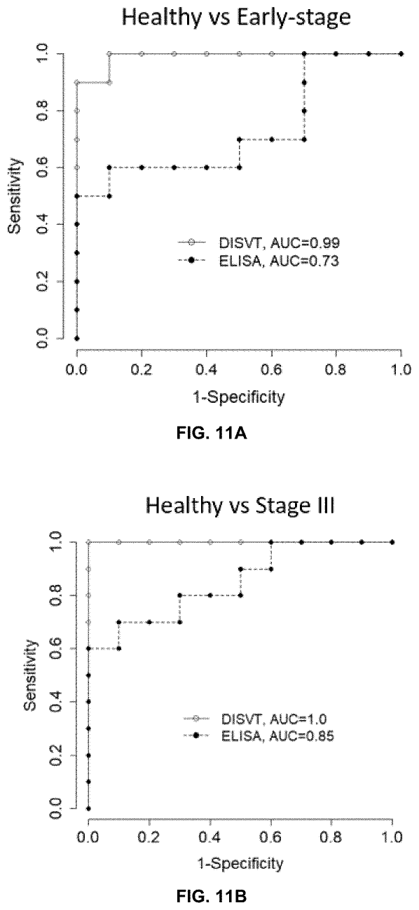

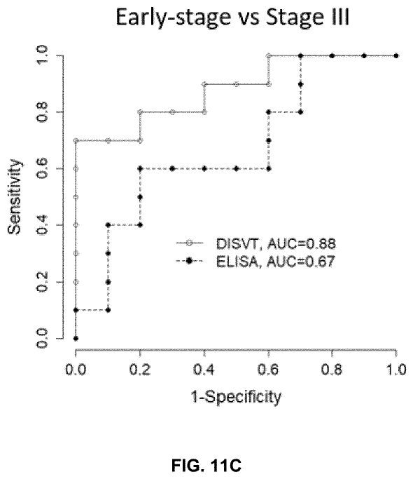

The diagnostic strength of EV HER2 expression for HER2-positive breast cancer with DISVT was further evaluated with a comparison to ELISA by the receiver operation characteristic (ROC) curves (FIGS. 11A-11C). In agreement with the ANOVA analysis, the DISVT was much more sensitive than ELISA in detecting early-stage breast cancer, with the area under curve (AUC)=0.99 for DISVT versus 0.73 for ELISA. DISVT was also more sensitive that ELISA in detecting stage III breast cancer, with AUC=1 for DISVT versus AUC=0.85 for ELISA. In addition, DISVT was more sensitive than ELISA in differentiating early-stage from stage III breast cancer, with AUC=0.88 for DISVT versus AUC=0.67 for ELISA.

The difference of the detection sensitivity between DISVT and ELISA is due to the difference in the methodology for molecular marker detection and signal collection. The DISVT detects HER2 on individual EVs, with the detection sensitivity down to single molecule as the dark field image can detect AuNPs at single particle level. It detects target specific EVs at single vesicle level in the sea of normal EVs. However, the ELISA detects collective HER2 signals from a bulk of EVs. If the number of HER2-positive EVs is low, the HER2 signals from the bulk EVs are not sufficiently high to be detected by the ELISA plate reader. Thus, DISVT is superior to detect rare disease signals in the presence of normal backgrounds by identification and detection of target specific individual EVs.

### OTHER EMBODIMENTS

From the foregoing description, it will be apparent that variations and modifications may be made to the invention described herein to adopt it to various usages and conditions. Such embodiments are also within the scope of the following claims. Additional variations and modifications may be made based on the teachings of the pending patent application published as US 2020/0191778 A1, which is incorporated by reference.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for characterizing membrane bound vesicles present in a biological sample, the method comprising:
a) contacting a biological sample comprising a membrane bound vesicle comprising a lipophilic dye with a gold-coated substrate comprising a first capture molecule fixed to a surface of a substrate, wherein the first capture molecule specifically binds a first surface protein present on the surface of the membrane bound vesicle, thereby fixing the membrane bound vesicle to the surface of the substrate;
b) contacting the membrane bound vesicle with a second capture molecule, wherein the second capture molecule is fixed to the surface of a nanoparticle, wherein the second capture molecule specifically binds a surface marker of interest on the membrane bound vesicle;
c) subjecting the membrane bound vesicle to fluorescence imaging and dark field imaging, wherein the fluorescence imaging localizes vesicles on the slide and the dark field imaging characterizes the presence or absence of the surface marker of interest on the vesicles, thereby obtaining dark field images and fluorescent images;
d) computationally analyzing overlap of the fluorescence images and the dark field images to identify vesicles having or lacking the surface marker of interest; and
e) extracting pixel intensity of the nanoparticles from the images to characterize an expression profile of a protein of interest at a location of the vesicle, thereby obtaining a protein expression profile for the protein of interest present in the biological sample and quantifying target-specific vesicle subtypes.

2. The method of claim 1, further comprising computationally analyzing the images and/or protein expression profiles to determine the fraction of vesicles that are positive or negative for the surface marker of interest and the level of expression of the surface marker of interest on the positive or negative vesicles.

3. A method for characterizing exosomes present in a biological sample, the method comprising:
a) contacting a liquid biological sample comprising one or more exosomes comprising a lipophilic dye with a gold-coated multi-well slide comprising an antibody or antigen binding fragment thereof fixed to a surface of the slide, thereby fixing the exosomes to the surface of the slide;
b) contacting the exosomes with a second antibody or second antigen binding fragment thereof fixed to a surface of a metal nanoparticle, wherein the second antibody or second antigen binding fragment thereof specifically binds a polypeptide surface marker of interest;
c) subjecting the exosomes to fluorescence imaging and dark field imaging, wherein the fluorescence imaging localizes the exosomes on the slide and the dark field imaging characterizes the presence or absence of the polypeptide surface marker of interest on the exosomes, thereby obtaining dark field and fluorescent images;
d) computationally analyzing overlap of the fluorescence images and the dark field images to identify exosomes having or lacking the surface marker of interest; and
e) extracting pixel intensity of the metal nanoparticles from the images to characterize an expression profile of a protein of interest at a location of the exosome, thereby obtaining the protein expression profile.

4. The method of claim 3, wherein the protein expression profile and/or images are further computationally analyzed to determine the fraction of vesicles that are positive for the surface marker of interest and the level of expression of the marker of interest on marker positive vesicles and marker negative vesicles.

5. The method of claim 3, wherein the metal nanoparticle comprises silver, gold, copper, titanium, platinum, zinc, iron, or magnesium.

6. The method of claim 1, wherein the biological sample is blood, plasma, serum, cerebrospinal fluid, ascites, or culture media.

7. The method of claim 1, wherein the first capture molecule and/or second capture molecule is an antibody, aptamer, or other molecule that specifically binds an antigen present on the surface of an extracellular vesicle.

8. The method of claim 1, wherein the second capture molecule is an antibody or antigen binding fragment thereof that specifically binds an ALIX, TSG101, CD81, CD63, or CD9 polypeptide.

9. The method of claim 1, wherein the lipophilic dye comprises a lipophilic molecule having an alkyl chain and an affinity for a lipid bilayer of an extracellular vesicle.

10. The method of claim 9, wherein the lipophilic molecule comprises 1,2-distearoyl-sn-glycerol-3-phosphoethanoloamine conjugated polyethylene glycol thiol (DSPE-PEG-SH).

11. The method of claim 9, wherein the lipophilic dye comprises cholesterol-polyethylene glycol-Cy5 (CLS-PEG-Cy5).

12. The method of claim 1, wherein the fluorescence is generated using a laser.

13. The method of claim 12, wherein the laser emits a wavelength of light between 600-700 nanometers.

14. The method of claim 1, wherein the nanoparticle is bound to the vesicle or exosome via an antibody linked to the nanoparticle, wherein the antibody specifically binds to a marker present on the vesicle or exosome.

15. The method of claim 14, wherein the vesicle or exosome comprises a polypeptide surface marker selected from the group consisting of: HER2, CD44, CLDN4, EPCAM, CD151, LGALS3BP, HIST2H2BE, or HIST2H2BF.

16. The method of claim 15, wherein detection of the marker is indicative of disease.

17. The method of claim 1, wherein the lipophilic dye comprises cholesterol-polyethylene glycol-Cy5.

18. The method of claim 1, wherein the second capture molecule or antibody is linked to the nanoparticle by NHS-PEG-SH.

19. A dual fluorescence and dark field microscopic imaging system, the system comprising:
a dark field microscope comprising a halogen illumination lamp configured for dark-field white light illumination and an objective lens positioned over a sample field; and
an excitation laser positioned in an angled direction, wherein the angle is at least about 30-60 degrees relative to the sample field, wherein when a sample present in the sample field is illuminated by the lamp and excited by the laser, signals are transmitted to an imaging camera and a spectrometer.

20. The system of claim 19, wherein the system comprises an excitation laser angled at about 45 degrees relative to the sample field.
